(19) United States
(12) Patent Application Publication
(10) Pub. No.: US 2012/0003241 A1
(43) Pub. Date: Jan. 5, 2012

(54) VACCINE AGAINST BOTULISM

(75) Inventors: Mingtao Zeng, Pittsford, NY (US); Michael E. Pichichero, Rochester, NY (US); Qingfu Xu, Rochester, NY (US)

(73) Assignee: UNIVERSITY OF ROCHESTER, Rochester, NY (US)

(21) Appl. No.: 12/672,604

(22) PCT Filed: Aug. 8, 2008
(86) PCT No.: PCT/US08/72585

§ 371 Date: Jul. 19, 2010

Related U.S. Application Data
(60) Provisional application No. 60/954,921, filed on Aug. 9, 2007.

(51) Int. Cl. A61K 39/395; C12N 15/62; C12N 15/63; A61P 37/04; C07K 19/00; A61K 39/08; C07K 16/12; A61P 31/04; C12N 15/31; C12N 5/10

(52) U.S. Cl. 424/167.1; 536/23.7; 536/23.4; 435/320.1; 435/325; 530/350; 424/239.1; 424/192.1; 530/389.5

(57) ABSTRACT

The invention relates to novel DNA and protein vaccines against Clostridium botulinum. The DNA vaccine includes a DNA molecule that includes a first segment encoding a fragment of a heavy chain region of a Clostridium botulinum neurotoxin, wherein the first segment is codon-enhanced to improve expression of the isolated DNA molecule in a mammalian host, and preferably a second segment that encodes a secretion signal peptide. The chimeric protein of the present invention includes the secretion signal peptide linked N-terminal of the fragment of a heavy chain region of a Clostridium botulinum neurotoxin. Use of these materials to raise antibodies, and to impart resistance against Clostridium botulinum to a mammal is also disclosed.

This application claims the priority benefit of U.S. Provisional Patent application Ser. No. 60/954,921, filed Aug. 9, 2007, which is hereby incorporated by reference in its entirety.

The present invention was made with government support under grant number R21AI055946 from the National Institute of Allergy and Infectious Diseases, National Institutes of Health (NIAID/NIH). The government has certain rights in this invention.

### FIELD OF THE INVENTION

The present invention relates to novel DNA and protein vaccines for use in inducing a protective immune response against Clostridium botulinum.

### BACKGROUND OF THE INVENTION

Botulism is a life-threatening neuroparalytic disease caused by botulinum neurotoxins (BoNTs), which are produced by one of the seven structurally similar Clostridium botulinum serotypes, designated A to G in which type C has two subtypes (C1 and C2). In addition, Clostridium baratii synthesizes only serotype F and Clostridium butyricum synthesizes only serotype E. As the concept of serotype implies, each of the toxins is immunologically distinct. The only exception to this general rule is serotypes C and D, which share significant cross-homology (Oguma et al., “Antigenic Similarity of Toxins Produced by Clostridium Botulinum Type C and D Strains,” Infect Immun 30(3):656-60 (1980)). BoNTs are the most poisonous substances known in nature. They may be used as bioterrorism agents or in biological warfare (Arnon et al., “Botulinum Toxin as a Biological Weapon,” Medical and Public Health Management. JAMA 285(8):1059-70 (2001)). Therefore, there is an urgent need for the development of effective vaccines to protect against botulism.

Currently, a pentavalent botulinum toxoid vaccine that may protect against BoNT serotypes A-E is available as an Investigational New Drugs (IND) (Wright et al., “Studies on Immunity to Toxins of Clostridium botulinum: V. Detoxification of Purified Type A and Type B Toxins, and the Antigenicity of Univalent and Bivalent Aluminium Phosphate Adsorbed Toxoids,” J Immunol 84:384-9 (1960); Fiock et al., “Studies on Immunity to Toxins of Clostridium botulinum: IX. Immunologic Response of Man to Purified Pentavalent ABCDE Botulinum Toxoid,” J Immunol 90:697-702 (1963)). However, there are several shortcomings with the toxoid vaccines. First, the cost of manufacturing is very high, because C. botulinum is a spore-former and a dedicated cGMP facility is required to manufacture a toxin-based product. The yields of toxin production from C. botulinum are relatively low, it is dangerous to produce them—as the toxoiding process involves handling large quantities of toxin, and the added safety precautions increase the cost of manufacturing. Second, the toxoid product for types A-E is in the form a crude extract of clostridial proteins that may influence immunogenicity or reactivity of the vaccine, and the type F toxoid is only partially purified. Third, residual formaldehyde (not to exceed 0.02%) and the preservative thimerosal (0.01%) are part of final product formulation. This increases the reactogenicity of the vaccine (Byrne et al., “Development of Vaccines for Prevention of Botulism,” Biochimie 82(9-10):955-66 (2000)).

A high sequence and structural homology exists between the clostridial neurotoxins produced by Clostridium tetani and C. botulinum. The successful demonstration that a C-fragment of tetanus toxin (TeNT) elicits protective immunity in animals has prompted the development of a new subunit vaccine against botulism (Helting et al., “Analysis of the Immune Response to Papain Digestion Products of Tetanus Toxin,” Acta Pathol Microbiol Immunol Scand [C], 92(1):59-63 (1984); Fairweather et al., “Immunization of Mice Against Tetanus with Fragments of Tetanus Toxin Synthesized in Escherichia coli,” Infect Immun 55(11):2541-5 (1987)). Evaluation of the immunogenicity of different regions of BoNTs has confirmed that the C-fragment of the BoNTs elicits protective immunity in animals (LaPenotiere et al., “Expression of a Large, Nontoxic Fragment of Botulinum Neurotoxin Serotype A and its Use as an Immunogen,” Toxicon 33(10):1383-6 (1995); Clayton et al., “Protective Vaccination With a Recombinant Fragment of Clostridium Botulinum Neurotoxin Serotype A Expressed From a Synthetic Gene in Escherichia Coli,” Infect Immun 63(7):2738-42 (1995); Dertzbaugh et al., “Mapping of Protective and Cross-Reactive Domains of the Type A Neurotoxin of Clostridium Botulinum,” Vaccine 14(16):1538-44 (1996); Lee et al. “C Terminal Half Fragment (50 kDa) of Heavy Chain Components of Clostridium Botulinum Type C and D Neurotoxins Can Be Used as an Effective Vaccine,” Microbiol Immunol 51(4):445-55 (2007); Webb et al., “Protection With Recombinant Clostridium Botulinum C1 and D Binding Domain Subunit (Hc) Vaccines Against C and D Neurotoxins,” Vaccine 16:16 (2007); Boles et al., “Recombinant C Fragment of Botulinum Neurotoxin B Serotype (rBoNTB (HC)) Immune Response and Protection in the Rhesus Monkey,” Toxicon 47(8):877-84 (2006)). Therefore, subsequent efforts to develop vaccine candidates to protect against BoNTs forthwith may focus on the H_{C} region of BoNTs (Baldwin et al., “Characterization of the Antibody Response to the Receptor Binding Domain of Botulinum Neurotoxin Serotypes A and E,” Infect Immun 73(10):6998-7005 (2005)). Because the C-domains of the BoNTs are responsible for receptor binding, host immune response against these domains may prevent the BoNTs from gaining access into target cells. Research on peptide-based vaccines has shown several synthetic peptides elicit antibody and T-cell responses in two different strains of mice (BALB/c and SJL) that cross-react with the H_{C} region of BoNT/A. These experiments show the feasibility of developing a synthetic vaccine that could protect against botulinum neurotoxin intoxication (Byrne et al., “Development of Vaccines for Prevention of Botulism,” Biochimie 82(9-10):955-66 (2000); Atassi et al., Mapping of the Antibody-Binding Regions on Botulinum Neurotoxin H-Chain Domain 855-1296 With Antitoxin Antibodies From Three Host Species,” J Protein Chem, 15(7):691-700 (1996); Atassi et al., “Structure, Activity, and Immune (T and B cell) Recognition of Botulinum Neurotoxins,” Crit Rev Immunol 19(3):219-60 (1999); Oshima et al., “Immune Recognition of Botulinum Neurotoxin Type A: Regions Recognized by T cells and Antibodies Against the Protective H(C) Fragment (Residues 855-1296) of the Toxin,” Mol Immunol 34(14):1031-40 (1997); Oshima et al., “Antibodies and T cells Against Synthetic Peptides of the C-Terminal Domain (Hc) of Botulinum Neurotoxin Type A and Their Cross-Reaction With Hc,” Immunol Lett 60(1):7-12 (1998)). The employment of the non-toxic fragments of BoNTs as protective antigens also provides a significant safety profile advantage. However, expression and purification of recombinant fragments of BoNTs, and their subsequent formulation into a vaccine is costly.

Given the need for an effective vaccine against botulism, it would be desirable to develop a single-dose vaccine that provides long-lasting protective immunity against botulism. The present invention is directed to overcoming these and other deficiencies in the art.

### SUMMARY OF THE INVENTION

A first aspect of the present invention relates to an isolated DNA molecule that includes a first segment encoding a fragment of a heavy chain region of a Clostridium botulinum neurotoxin, wherein the first segment is codon-enhanced to improve expression of the isolated DNA molecule in a mammalian host.

A second aspect of the present invention relates to an expression vector or plasmid that includes an isolated DNA molecule according to the first aspect of the present invention operably coupled to one or more regulatory sequences that afford transcription of the isolated DNA molecule in the mammalian host. Also encompassed with this aspect of the present invention is a recombinant host cell that includes an expression vector or plasmid according to this aspect of the invention.

A third aspect of the present invention relates to a chimeric protein that includes a secretion signal peptide linked N-terminal of a fragment of a heavy chain region of a Clostridium botulinum neurotoxin.

A fourth aspect of the present invention relates to a vaccine that includes a pharmaceutically acceptable carrier and either (i) a DNA molecule according to the first aspect of the present invention or an expression vector according to the second aspect of the present invention; (ii) a chimeric protein according to the third aspect of the present invention; or a combination of (i) and (ii).

A fifth aspect of the present invention relates to a method of imparting resistance against a Clostridium botulinum neurotoxin to a mammal, which includes administering a vaccine according to the fourth aspect of the present invention to a mammal under conditions effective to induce a protective immune response against the Clostridium botulinum neurotoxin. A related aspect of the invention relates to a method of neutralizing a neurotoxin of the present invention.

A sixth aspect of the present invention relates to an isolated antibody raised against a chimeric protein according to the third aspect of the present invention, or an antibody binding fragment thereof. A pharmaceutical composition containing the antibody or binding fragments thereof is also encompassed by this aspect of the present invention.

A seventh aspect of the present invention relates to a hybridomas cell that expresses a monoclonal antibody according to the sixth aspect of the present invention.

An eighth aspect of the present invention relates to a method of treating a Clostridium botulinum infection that includes administering to a patient an antibody or antibody fragment thereof (or a pharmaceutical composition containing the same) according to the sixth aspect of the invention, wherein the administration thereof is carried out under conditions effective to neutralize a botulism neurotoxin, and thereby treat the Clostridium botulinum infection.

As demonstrated in the accompanying Examples, a single dose of an adenoviral vector encoding a codon-optimized fusion (chimeric) protein, containing an N-terminal secretion signal peptide and a fragment of a heavy chain region of a Clostridium botulinum neurotoxin, was sufficient to induce a protective immune response against the neurotoxin from which the heavy chain region was derived. Importantly, a single dose of 2×10⁷ pfu of Ad/opt-BoNT/C—H_{C}50 was sufficient to provide long-term protective immunity via either intramuscular or intranasal vaccination. This study is the first to demonstrate that a single genetic vaccination is able to provide long-lasting protection against botulism.

### BRIEF DESCRIPTION OF THE DRAWINGS

FIGS. 1A-C are graphs that illustrate anti H_{C}50 of BoNT/C response in vaccinated mice. Mice were inoculated with different doses of Ad/opt-BoNT/C—H_{C}50 in week 0, Serum samples were obtained at weeks 0, 2, 4, and 6. Anti-BoNT/C—H_{C}50 IgG (FIG. 1A), IgG1 (FIG. 1B) and IgG2a (FIG. 1C) antibody concentrations were measured by a quantitative ELISA kit (Bethyl, Montgomery, Tex.). Virus doses for groups I, II, and III were 10⁵, 10⁶, and 2×10⁷ pfu, respectively. Mean=X±SE (n=8). Values without the same letters (a, b, c, d) differ significantly in the same dosage groups (P<0.05). *P<0.05, **P<0.01.

FIGS. 2A-B are graphs that illustrate serum anti-BoNT/C neutralizing antibody titer assay. 50 μA of serum from each mouse in the same group were pooled 6 weeks after vaccination with Ad/opt-BoNT/C—H_{C}50 (8 mice/group). Sera were 1:4 diluted initially with Dulbecco's PBS and then in twofold series for determination of anti-BoNT/C neutralization titers. FIG. 2A shows mice survival rates after challenge with neutralized BoNT/C. FIG. 2B shows serum anti-BoNT/C neutralization titers (IU/ml, one IU is equal to 10,000×MLD₅₀). IM: vaccination (n=4).

FIG. 3 is a graph showing protection against active BoNT/C in mice vaccinated with adenoviral vector. Mice were vaccinated with different dosages of adenovirus-vectored vaccine Ad/opt-BoNT/C—H_{C}50 in week 0 and then challenged in week 7 with 100×MLD₅₀ BoNT/C. Ad/opt-BONT/C—H_{C}50-vaccinated groups: I, 10⁵ pfu; II, 10⁶ pfu; III, 2×10⁷ pfu; N.Con: negative control was inoculated with 2×10⁷ pfu of Ad/Null; P.Con: positive control group was vaccinated i.m. with 50 μl of the pentavalent (ABCDE) botulinum toxoid vaccine. (n for Groups III, II, I, N.Con, and P.Con are 12, 8, 8, 8, and 12, respectively.)

FIG. 4 is a graph illustrating the sustaining of antigen specific antibody responses after vaccination with the adenovirus-vectored vaccine in mice. Mice were inoculated i.m. with a single dose of 2×10⁷ pfu of Ad/opt-BONT/C—H_{C}50 (vaccination) or with Ad/Null (control) in week 0, Serum samples were obtained in weeks 11, 19, and 27 before challenging with BoNT/C. The anti-BoNT/C—H_{C}50 IgG antibody concentrations in sera were determined using a quantitative ELISA kit (Bethyl, Montgomery, Tex.). Mean=X±SD (n=7 or 8 in vaccination groups and n=4 in control groups).

FIGS. 5A-C are graphs illustrating the long-lasting protective immunity in vaccinated mice against BoNT/C challenge. Mice were injected i.m. with 2×10⁷ pfu of Ad/opt-BoNT/C—H_{C}50 or with Ad/Null in week 0 and then challenged with 100×MLD₅₀ BoNT/C in week 11 (FIG. 5A), week 19 (FIG. 5B), and week 27 (FIG. 5C). n=8 in experiment groups; n=4 in control groups.

FIGS. 6A-B are graphs illustrating the effect of pre-existing immunity to adenovirus on the efficacy of the adenovirus-vectored vaccine. FIG. 6A shows anti-adenovirus neutralizing antibodies in animals inoculated with adenovirus pre-vaccination. Mice were inoculated i.n. with 2×10⁷ pfu/mouse of wild-type human adenovirus serotype 5 in week 0, Serum samples were obtained in week 4 and the anti-Ad5 neutralizing antibody titers were subsequently measured. Mean=X±SE. IM group: the group that was subsequently vaccinated with Ad/opt-BONT/C—H_{C}50 in FIG. 6B; Ad/Null: the group that was subsequently injected with Ad/Null in FIG. 6B; Con: data were obtained from mouse sera before inoculation of WT Ad5 in FIG. 6B. In FIG. 6B, each mouse was inoculated i.n. with 2×10⁷ pfu of WT Ad5 in week 0 as described above, and then subsequently injected with 2×10⁷ pfu Ad/opt-BoNT/C—H_{C}50 in vaccination group or with Ad/Null in control group in week 4, and challenged with 100×MLD₅₀ BoNT/C in week 11. (n=8 in vaccination groups; n=4 in control groups.)

FIGS. 7A-C are graphs illustrating the serum antibody responses against BoNT/C—H_{C}50 in vaccinated mice. Mice were vaccinated intranasally with different doses of Ad/opt-BoNT/C—H_{C}50 (1×10⁵ to 2×10⁷ pfu/mouse) in week 0, Serum samples were obtained in weeks 0, 2, 4, and 6 to measure anti-BoNT/C—H_{C}50 IgG (FIG. 7A), IgG1 (FIG. 7B), and IgG2a (FIG. 7C) antibody concentrations by quantitative ELISA. Mean=X±SE (n=8).

FIG. 8 is a graph illustrating the sustaining of antigen-specific antibody responses after intranasal vaccination with the adenovirus-vectored vaccine in mice. Mice were intranasally inoculated with a single dose of 2×10⁷ pfu of Ad/opt-BONT/C—H_{C}50 (Vaccination group) or with Ad/Null (Control group) in week 0, Serum samples were obtained in weeks 11, 19, and 27 before challenging with active BoNT/C. The anti-BoNT/C—H_{C}50 IgG antibody concentrations in sera were determined by quantitative ELISA. Mean=X±SD (n=7 or 8 in vaccination groups, and n=4 in control groups).

FIGS. 9A-D are graphs illustrating mucosal antibody responses against BoNT/C—H_{C}50 in vaccinated mice. Mice were intranasally inoculated with a single dose of 2×10⁷ pfu of Ad/opt-BONT/C—H_{C}50 (Vaccination) or with Ad/Null (Control) in week O, Saliva, nasal and vaginal wash samples were collected in weeks 2 and 4. Anti-BoNT/C—H_{C}50 IgG (FIG. 9A), IgG1 (FIG. 9B), IgG2a (FIG. 9C), and IgA (FIG. 9D) concentrations were measured by quantitative ELISA. X=Mean±SD (n=8 in Vaccination group and n=4 in Control group).

FIGS. 10A-B are graphs illustrating the results of serum anti-BoNT/C neutralizing antibody titer assay. 50 μA of serum from each mouse in the same group were pooled 6 weeks after vaccination intranasally with Ad/opt-BoNT/C—H_{C}50 (8 mice/group). Sera were 1:4 diluted initially with Dulbecco's PBS and then in two fold series for determination of anti-BoNT/C neutralization titers. FIG. 10A shows mice survival rates after challenge with neutralized BoNT/C. FIG. 10B shows serum anti-BoNT/C neutralization titers (IU/ml). One IU is equal to 10,000×MLD₅₀. CON, control; IMM: vaccination. (n=4).

FIG. 11A-D are graphs illustrating the protection against active BoNT/C challenge in vaccinated mice. Mice were intranasally inoculated with Ad/Null (N. Control group) or 1×10⁵-2×10⁷ of Ad/opt-BoNT/C—H_{C}50 in week 0, and then challenged with 100×MLD₅₀ of BoNT/C in weeks 7 (FIG. 11A), 11 (FIG. 11B), 19 (FIG. 11C), and 27 (FIG. 11D). Adenovirus dosages for Dose I, II, III, and N. Con are 1×10⁵, 1×10⁶, 2×10⁷, and 2×10⁷ pfu/mouse, respectively. (n=4 for N. Control groups; n=8 for vaccination groups.)

FIG. 12 is a graph illustrating the Botulinum neurotoxin dose-dependent protection in mice vaccinated with adenovirus-vectored vaccine. Mice were intranasally vaccinated with 2×10⁷ pfu of Ad/opt-BoNT/C—H_{C}50 in week 0 and then challenged in week 4 with 10³-10⁵×MLD₅₀ of BoNT/C. Ad/Null, negative control, animals were inoculated intranasally with 2×10⁷ pfu of Ad/Null and challenged with 10³×MLD₅₀ of BoNT/C. (n for Ad/Null, vaccinated groups 10³ MLD₅₀, 10⁴ MLD₅₀, 10⁵ MLD₅₀ are 4, 8, 8, 7, respectively.)

FIG. 13 is a graph illustrating the anti-adenovirus neutralizing antibody response in mice inoculated with adenovirus pre-vaccination. Mice were intranasally inoculated with 2×10⁷ pfu of wild-type human adenovirus serotype 5 in week 0, Serum samples were obtained in week 4. Sera from 2 mice in the same group were pooled and the anti-Ad5 neutralizing antibody titers of the serum pools were subsequently measured. Mean=X±SE. Ad/Hc50: the group were subsequently vaccinated with Ad/opt-BoNT/C—H_{C}50 (FIG. 14); Ad/Null: the group were subsequently inoculated with Ad/Null (FIG. 14); N. Con: data were obtained from mouse pre-inoculation of adenovirus. (n=4.)

FIG. 14 is a graph illustrating the effect of pre-existing immunity to adenovirus on the efficacy of the adenovirus-vectored mucosal vaccine. Each mouse was inoculated intranasally with or without 2×10⁷ pfu of Ad5 in week 0 as shown in FIG. 13, then subsequently inoculated intranasally with 2×10⁷ pfu of Ad/opt-BoNT/C—H_{C}50 in vaccination group or with Ad/Null in control group in week 4, and challenged with 100×MLD₅₀ BoNT/C in week 11. (n=8 in experiment groups; n=4 in control group.)

### DETAILED DESCRIPTION OF THE INVENTION

The present invention relates to DNA and/or protein vaccines against botulism neurotoxin. The vaccines of the present invention are capable of inducing, with a single vaccination, a high titer of neutralizing antibodies against the botulism neurotoxin.

Neutralizing antibody titers are defined as the maximum number of IU of antitoxin per ml of serum, resulting in 100% survival after challenge, where one IU of botulinum neurotoxin antitoxin neutralizes 10,000×MLD₅₀ neurotoxin (Byrne et al., “Purification, Potency, and Efficacy of the Botulinum Neurotoxin Type A Binding Domain from Pichia pastoris as a Recombinant Vaccine Candidate.” Infect Immun 66(10):4817-22 (1998); Nowakowski et al., “Potent Neutralization of Botulinum Neurotoxin by Recombinant Oligoclonal Antibody,” Proc Natl Acad Sci USA 99:11346-11350 (2002), each of which is hereby incorporated by reference in its entirety). As used herein, a high titer of neutralizing antibodies refers to at least about 1 IU/ml, more preferably at least about 6 IU/ml, and most preferably at least about 10 IU/ml.

The vaccines of the present invention are suitable for use in any mammal including, without limitation, humans and nonhuman primates, such as chimpanzees and other apes and monkey species; farm animals including cattle, sheep, pigs, goats and horses, etc.; domestic animals including cats and dogs; laboratory animals including rodents such as mice rats, and guinea pigs, and the like. The mammal can be of any age or sex. Thus, adults and post-natal (newborn) subjects, as well as fetuses, are intended to be covered.

The DNA vaccine involves the use of a DNA molecule that contains a first nucleotide sequence encoding a fragment of a heavy chain region of a Clostridium botulinum neurotoxin, wherein the nucleotide sequence is codon-enhanced to improve expression of the DNA molecule in a mammalian host. The DNA molecule also preferably contains a second nucleotide sequence encoding a secretion signal peptide. The second nucleotide sequence is preferably located 5′ of the first nucleotide sequence, affording expression of a chimeric protein that includes an N-terminal secretion signal peptide and the fragment of a heavy chain region of a Clostridium botulinum neurotoxin (BoNT).

The BoNT from which the fragment is derived can be any one or more of neurotoxin A (see Genbank Accession No. X52066, which is hereby incorporated by reference in its entirety), neurotoxin B (see Genbank Accession No. M81186, which is hereby incorporated by reference in its entirety), neurotoxin C (see Genbank Accession No. D90210, which is hereby incorporated by reference in its entirety), neurotoxin D (see Genbank Accession No. X54254, which is hereby incorporated by reference in its entirety), neurotoxin E (see Genbank Accession No. X62089, which is hereby incorporated by reference in its entirety), neurotoxin F (see Genbank Accession No. M92906, which is hereby incorporated by reference in its entirety), or neurotoxin G (see Genbank Accession No. X74162, which is hereby incorporated by reference in its entirety).

The fragment of the BoNT heavy chain region should be non-toxic and antigenic, and capable of eliciting immunity responses against botulism. Preferably, the fragment of the heavy chain region is a C-terminal fragment that is about 50 kDa (referred to hereinafter as “H_{C}50” subunit or fragment). The H_{C}50 fragments of BoNTs are known to possess these attributes, i.e., non-toxic, antigenic, and capable of eliciting immunity responses against botulism (Byrne et al., “Development of Vaccines for Prevention of Botulism,” Biochimie 82:955-966 (2000); Webb et al., “Protection with Recombinant Clostridium botulinum C1 and D Binding Domain Subunit (Hc) Vaccines Against C and D Neurotoxins,” Vaccine 25:4273-4282 (2007); Byrne et al., “Purification, Potency, and Efficacy of the Botulinum Neurotoxin Type A Binding Domain from Pichia pastoris as a Recombinant Vaccine Candidate,” Infect Immun 66:4817-4822 (1998); Atassi et al., “Structure, Activity, and Immune (T and B cell) Recognition of Botulinum Neurotoxins,” Crit Rev Immunol 19:219-260 (1999), each of which is hereby incorporated by reference in its entirety.)

The DNA molecule can also include multiple open reading frames that afford expression of any combinations of the H_{C}50 fragments of BoNTs A-G, thus affording a multivalent vaccine.

As noted above, the first nucleotide sequence is preferably codon-optimized. Codon-optimization of the nucleotide sequence encoding the H_{C}50 fragment of BoNTs is believed to afford high expression levels of the expressed polypeptide, which in turn affords an immune response that produces a high titer of neutralizing antibodies.

According to one embodiment of the present invention, a codon-optimized DNA sequence encoding the H_{C}50 fragment of BoNT/C is shown in Table 1 below. The codon-optimized DNA sequence is SEQ ID NO: 2, which is compared to the native DNA sequence of SEQ ID NO: 1. Differences between these two redundant sequences is shown by nucleotide symbols in bold typeface in SEQ ID NO: 2. The encoded H_{C}50 fragment of BoNT/C has the amino acid sequence of SEQ ID NO: 3.

According to another embodiment of the present invention, a codon-optimized DNA sequence encoding the H_{C}50 fragment of BoNT/A is shown in Table 2 below. The codon-optimized DNA sequence is SEQ ID NO: 5, which is compared to the native DNA sequence of SEQ ID NO: 4. Differences between these two redundant sequences is shown by nucleotide symbols in bold typeface in SEQ ID NO: 5. The encoded H_{C}50 fragment of BoNT/A has the amino acid sequence of SEQ ID NO: 6.

The second nucleotide sequence can encode any suitable secretion signal peptide that affords secretion of the chimeric protein in mammalian cells. The secretion signal peptide should not interfere with the antigenicity of the encoded H_{C}50 BoNT fragment. Exemplary secretion signal peptides include, without limitation, human tissue plasminogen activator, human serum albumin, human IL-3, human growth hormone, etc.

The 25-amino acid secretion signal of human tissue plasminogen activator and its encoding nucleotide sequence are reported at Genbank Accession Nos. BC002795 and AAH02795, each of which is hereby incorporated by reference in its entirety.

The 24-amino acid secretion signal peptide of human serum albumin and its encoding nucleotide sequence are reported at Genbank Accession Nos. AAA98797 and M12523, each of which is hereby incorporated by reference in its entirety.

The 19-amino acid secretion signal peptide of human IL-3 and its encoding nucleotide sequence are reported at Genbank Accession Nos. NP_{—}000579 and NM_{—}000588, each of which is hereby incorporated by reference in its entirety.

The 26-amino acid secretion signal peptide of human growth hormone and its encoding nucleotide sequence are reported at Genbank Accession Nos. AAA72555 and M14422 (synthetic construct), each of which is hereby incorporated by reference in its entirety.

The nucleic acid molecules encoding the various polypeptide components of a chimeric protein can be ligated together along with appropriate regulatory elements that provide for expression of the chimeric protein. Typically, the nucleic acid construct encoding the chimeric protein can be inserted into any of the many available expression vectors and cell systems using reagents that are well known in the art.

For purposes of preparing the DNA vaccine of the present invention, the DNA molecule encoding the chimeric protein of the present invention is operably coupled to regulatory elements that are operable in mammalian systems. For purposes of preparing the chimeric protein, as the primary antigen of a protein-based vaccine, the DNA molecule encoding the chimeric protein can be operably coupled to regulatory elements that are operable in the desired eukaryotic or prokaryotic host cells in which recombinant expression of the chimeric protein is intended.

Referring now to the materials suitable for us in the DNA vaccine, the recombinant gene includes, operatively coupled to one another, an upstream promoter operable in mammalian cells and optionally other suitable regulatory elements (i.e., enhancer or inducer elements), the coding sequence that encodes the BoNT fragment, and a downstream transcription termination region. The promoter is preferably a constitutive promoter. Common promoters operable in mammalian cells include, without limitation, SV40, MMTV, metallothionein-1, adenovirus Ela, CMV immediate early, immunoglobulin heavy chain promoter and enhancer, and RSV-LTR promoters. Any suitable transcription termination region can be used, e.g., SV40 polyadenylation signal.

The DNA sequences of these various regions can be cloned into a shuttle or transfer vector using standard cloning procedures known in the art, including restriction enzyme cleavage and ligation with DNA ligase as described by Sambrook et al., Molecular Cloning: A Laboratory Manual, Second Edition, Cold Spring Harbor Press, NY (1989), and Ausubel et al., Current Protocols in Molecular Biology, John Wiley & Sons, New York, N.Y. (1989), which are hereby incorporated by reference in their entirety. U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. Thereafter, the recombinant gene can be similarly excised an inserted into a infective transformation vector or, alternatively, naked DNA or a recombinant plasmid can be used in combination with a non-infective delivery vehicle.

Any suitable viral or infective transformation vector can be used. Preferably, the infective transformation vector is replication-incompetent, and the vector itself is produced in a cell line that supplies any missing proteins suitable for production of the vector capable of transfecting cells with the recombinant transgene.

Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, “Development of Adenovirus Vectors for Expression of Heterologous Genes,” Biotechniques 6:616-627 (1988); Rosenfeld et al., “Adenovirus-Mediated Transfer of a Recombinant α1-Antitrypsin Gene to the Lung Epithelium in vivo,” Science 252:431-434 (1991); PCT Publication No. WO 93/07283; PCT Publication No. WO 93/06223; and PCT Publication No. WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety.

Adeno-associated viral gene delivery vehicles can be constructed and used to deliver into cells a recombinant gene encoding a desired nucleic acid. The use of adeno-associated viral gene delivery vehicles in vivo is described in Flotte et al., “Stable in vivo Expression of the Cystic Fibrosis Transmembrane Conductance Regulator with an Adeno-associated Virus Vector,” Proc. Nat'l Acad. Sci. USA 90:10613-10617 (1993); and Kaplitt et al., “Using Adeno-associated Virus Vectors in the Mammalian Brain,” Nature Genet. 8:148-153 (1994), each of which is hereby incorporated by reference in its entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, each of which is hereby incorporated by reference in its entirety.

As noted above, viral vectors have been successfully employed in order to increase the efficiency of introducing a recombinant vector into suitably sensitive host cells. Therefore, viral vectors are particularly suited for use in the present invention, including any adenoviral, retroviral, lentiviral, or adeno-associated viral vectors described above or known in the art. Current research in the field of viral vectors is producing improved viral vectors with high-titer and high-efficiency of transduction in mammalian cells (see, e.g., U.S. Pat. No. 6,218,187 to Finer et al., which is hereby incorporated by reference in its entirety). Such vectors are suitable in the present invention, as is any viral vector that includes a combination of desirable elements derived from one or more of the viral vectors described herein. It is not intended that the expression vector be limited to a particular viral vector.

Certain “control elements” or “regulatory sequences” are also incorporated into the vector-construct. The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Not all of these control elements need always be present so long as the selected coding sequence is capable of being replicated, transcribed, and translated in an appropriate host cell. Some of these control elements have been described above.

Following transfection of an appropriate host with the viral vector of the present invention, the virus is propagated in the host and collected. Generally, this involves collecting the cell supernatants at periodic intervals, and purifying the viral plaques from the crude lysate using techniques well-known in the art, for example, cesium chloride density gradient. The titer (pfu/ml) of the virus is determined, and can be adjusted up (by filtration, for example) or down (by dilution with an appropriate buffer/medium), as needed. In the present invention, typical Ad titers are in the range of 10⁶-10¹² pfu/ml.

Infective transformation vectors that contain a recombinant gene of the present invention can be presented for administration to a mammal in a pharmaceutical composition that includes a suitable carrier. Typically, DNA vaccines containing infective transformation vectors include a physiologically acceptable solution, such as, but not limited to, sterile saline or sterile buffered saline. Alternatively, the DNA may be associated with an adjuvant known in the art to boost immune responses, such as a protein or other carrier. Agents that assist in the cellular uptake of DNA, such as, but not limited to calcium ion, may also be used to advantage. These agents are generally referred to as transfection facilitating reagents and pharmaceutically acceptable carriers.

The infective transformation vectors are preferably administered in an effective amount to induce, with a single dose, a high titer of neutralizing antibodies. Dosages of the recombinant virus will depend primarily on factors, such as the condition being treated, the selected fusion protein, the age, weight, and health of the patient, and may thus vary among patients. A therapeutically effective human dosage of the viruses of the present invention is believed to be in the range of about 5 ml of saline solution containing concentrations of from about 10⁶ pfu/ml to 2.5×10¹² pfu/ml virus of the present invention. The dosage can be adjusted to balance the therapeutic benefit against any side effects. The levels of expression of the selected gene can be monitored to determine the selection, adjustment, or frequency of dosage administration. If required, a boost can be administered following a suitable period of delay to maximize the immune response against the botulism neurotoxin.

Any suitable mode of delivering an infective transformation vector is contemplated. Exemplary modes of delivery include, without limitation, intradermal or transdermal introduction; impression though the skin; intralesionally; via intramuscular, intraperitoneal, intravenous, intraarterial, or subcutaneous injection; via inhalation, and application to mucous membranes such as via intranasal delivery; orally; parenterally; implantation; and by intracavitary or intravesical instillation.

As noted above, non-infective DNA vaccines are also contemplated. These modes of administration encompass the use of naked DNA with or without an uptake agent, DNA bioconjugates, as well as DNA administered via a transfection agent. Preferably, the recombinant gene is present in the form of a non-infective DNA plasmid.

The DNA can be formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI (PEG-PEI) derivatives thereof (see, e.g., Furgeson et al., “Modified Linear Polyethylenimine—Cholesterol Conjugates for DNA Complexation,” Bioconjugate Chem. 14:840-847 (2003); Kunath et al., “The Structure of PEG-Modified Poly(Ethylene Imines) Influences Biodistribution and Pharmacokinetics of Their Complexes with NF-κB Decoy in Mice,” Pharmaceutical Res 19:810-817 (2002); Choi et al., “Effect of Poly(ethylene glycol) Grafting on Polyethylenimine as a Gene Transfer Vector in vitro,” Bull. Korean Chem. Soc. 22:46-52 (2001); Bettinger et al., “Size Reduction of Galactosylated PEI/DNA Complexes Improves Lectin-Mediated Gene Transfer into Hepatocytes,” Bioconjugate Chem. 10:558-561 (1999); Peterson et al., “Polyethylenimine-graft-Poly(ethylene glycol) Copolymers: Influence of Copolymer Block Structure on DNA Complexation and Biological Activities as Gene Delivery System,” Bioconjugate Chem. 13:845-854 (2002); Erbacher et al., “Transfection and Physical Properties of Various Saccharide, Poly(ethylene glycol), and Antibody-Derivatized Polyethylenimines (PEI),” J. Gene Medicine Preprint 1(2):210-222 (1999); Godbey et al., “Tracking the Intracellular Path of Poly(ethylenimine)/DNA Complexes For Gene Delivery,” Proc Natl Acad Sci USA 96:5177-5181 (1999); Godbey et al., “Poly(ethylenimine) and Its Role in Gene Delivery,” J Controlled Release 60:149-160 (1999); Diebold et al., J Biol Chem 274:19087-19094 (1999); Thomas et al., “Enhancing Polyethylenimine's Delivery of Plasmid DNA into Mammalian Cells,” Proc Natl Acad Sci USA 99:14640-14645 (2002); and U.S. Pat. No. 6,586,524 to Sagara, each of which is hereby incorporated by reference in its entirety.

The DNA molecule can also be present in the form of a bioconjugate, for example a nucleic acid conjugate as described in U.S. Pat. No. 6,528,631, U.S. Pat. No. 6,335,434, U.S. Pat. No. 6,235,886, U.S. Pat. No. 6,153,737, U.S. Pat. No. 5,214,136, or U.S. Pat. No. 5,138,045, each of which is hereby incorporated by reference in its entirety.

The recombinant DNA molecule can also be administered via a liposomal delivery mechanism. Basically, this involves providing a liposome which includes the DNA molecule (or plasmid) to be delivered, and then contacting a cell with the liposome under conditions effective for delivery of the DNA (or plasmid) into the cell. The liposomal delivery system can be made to accumulate at a target organ, tissue, or cell via active targeting (e.g., by incorporating an antibody or hormone on the surface of the liposomal vehicle). This can be achieved using antibodies specific for an appropriate cell marker.

Different types of liposomes can be prepared according to Bangham et al., “Diffusion of Univalent Ions Across the Lamellae of Swollen Phospholipids,” J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

These liposomes can be produced such that they contain, in addition to the DNA payload, other therapeutic agents, such as immune-enhancing agents, e.g., IL-2 or interferon alpha or GM-CSF), which would also be released at the target site (Wolff et al., “The Use of Monoclonal Anti-Thy1 IgG1 for the Targeting of Liposomes to AKR-A Cells in vitro and in vivo,” Biochem. et Biophys. Acta 802:259 (1984), which is hereby incorporated by reference in its entirety).

The amount of expressible DNA to be introduced into a vaccine recipient will depend partially on the strength of the promoters used and on the immunogenicity of the expressed gene product. In general, an immunologically or prophylactically effective dose of about 1 ng to 100 mg, and preferably about 10 μg to 300 μg of a plasmid vaccine vector is administered directly into tissue. The non-infective DNA vaccines are also intended to be administered in a physiologically acceptable solution, such as, but not limited to, sterile saline or sterile buffered saline. The DNA may also be associated with an adjuvant known in the art to boost immune responses, such as a protein or other carrier. Agents that assist in the cellular uptake of DNA, such as, but not limited to calcium ion, may also be used to advantage. These agents are generally referred to as transfection facilitating reagents and pharmaceutically acceptable carriers. Any suitable mode of delivering the non-infective DNA is contemplated, including those identified above for infective transformation.

Finally, the use of vaccines comprising the chimeric protein of the present invention is also contemplated. Preferably, the chimeric protein includes the N-terminal secretion signal linked as an in-frame gene fusion to the HC₅₀ BoNT fragment. The DNA encoding the chimeric protein is preferably introduced into a recombinant host cell using a suitable vector, after which the protein is expressed, and then recovered and purified before being presented in a pharmaceutical formulation suitable for administration.

Suitable vectors include, but are not limited to, the following viral vectors such as baculovirus lambda vector system gt1 1, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK +/−or KS +/−(see “Stratagene Cloning Systems” Catalog (1993) from Stratagene, La Jolla, Calif., which is hereby incorporated by reference in its entirety), pQE, pIH821, pGEX, pET series (see Studier et. al., “Use of T7 RNA Polymerase to Direct Expression of Cloned Genes,” Gene Expression Technology vol. 185 (1990), which is hereby incorporated by reference in its entirety), and any derivatives thereof.

The DNA sequences can be cloned into the vector using standard cloning procedures known in the art, including restriction enzyme cleavage and ligation with DNA ligase as described by Sambrook et al., Molecular Cloning: A Laboratory Manual, Second Edition, Cold Spring Harbor Press, NY (1989), and Ausubel et al., Current Protocols in Molecular Biology, John Wiley & Sons, New York, N.Y. (1989), which are hereby incorporated by reference in their entirety. Recombinant molecules, including plasmids, can be introduced into cells via transformation, particularly transduction, conjugation, mobilization, or electroporation. Once these recombinant plasmids are introduced into unicellular cultures, including prokaryotic organisms and eukaryotic cells, the cells are grown in tissue culture and vectors can be replicated.

Any number of vector-host combinations can be employed, including plasmids and bacterial host cells, yeast vectors and yeast hosts, baculovirus vectors and insect host cells, vaccinia virus vectors and mammalian host cells, etc.

As noted above, transcription of DNA is dependent upon the presence of a promoter, which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells. A number of promoters suitable for expression in eukaryotes and prokaryotes are well known in the art, any of which can be utilized.

The promoter used for expression of the above-identified proteins or polypeptide fragments thereof can be a constitutive promoter, which directs expression continually, or an inducible promoter, which is capable of directly or indirectly activating transcription of one or more DNA sequences or genes in response to an inducer (whereas, in the absence of an inducer the DNA sequences or genes will not be transcribed). In addition, any enhancer or inducer elements can be included to generate the level and control over expression of the transgene.

The DNA construct can also include an operable 3′ regulatory region, selected from among those which are capable of providing correct transcription termination and polyadenylation of mRNA for expression in the host cell of choice, operably linked to a DNA molecule which encodes for a protein of choice.

One alternative to the use of prokaryotic host cells is the use of eukaryotic host cells, such as yeast or mammalian cells, which can also be used to recombinantly produce the various proteins or polypeptide fragments thereof as noted above. Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-1, NIH3T3 (ATCC No. CRL 1658), and CNS1 cells. Suitable expression vectors for directing expression in mammalian cells generally include a promoter, as well as other transcription and translation control sequences known in the art and noted above for the infective transformation systems.

Once the DNA construct of the present invention has been prepared, it is ready to be incorporated into a host cell. Recombinant molecules can be introduced into cells via transformation, particularly transfection, lipofection, transduction, conjugation, mobilization, electroporation, or infection (e.g., with a viral vector). Accordingly, another aspect of the present invention relates to a method of making a recombinant host cell. Basically, this method is carried out by transforming a host cell with a DNA construct of the present invention under conditions effective to yield transcription of the DNA molecule in the host cell.

Once the host cell has been prepared, the chimeric can be expressed and recovered in a substantially pure form. In a particular embodiment, the substantially pure chimeric protein is at least about 80% pure, more preferably at least 90% pure, most preferably at least 95% pure. A substantially pure chimeric protein can be obtained by conventional techniques well known in the art. Given its secretion signal, the substantially pure chimeric protein is secreted into the growth medium of recombinant host cells. The medium can be recovered and then subjected to gel filtration in an appropriately sized dextran or polyacrylamide column to separate the chimeric protein from debris and other secreted proteins. If necessary, a protein fraction containing the substantially pure chimeric protein may be further purified by high performance liquid chromatography (“HPLC”).

The chimeric protein, once it has been recovered in substantially pure form, can be formulated into a vaccine that includes a pharmaceutically acceptable carrier, typically sterile saline or sterile buffered saline, and any suitable adjuvants such as those described above. In general, an immunologically or prophylactically effective dose of about 10 μg to 100 mg, and preferably about 100 μg to 1 mg of chimeric protein is administered directly into tissue. Any suitable mode of delivering the chimeric protein vaccine is contemplated, including those identified above for DNA-based vaccines.

From the foregoing, it should be appreciated that the present invention also relates to a method of imparting resistance against Clostridium botulinum (or botulism neurotoxin) to a mammal. This method is carried out by administering to a mammal a DNA or chimeric protein vaccine of the present invention under conditions effective to induce a protective immune response against Clostridium botulinum. As demonstrated by the accompanying Examples, a single dose of an infective adeno-virus vector of the present invention is capable of inducing long-term protection (for more than 6 months) against BoNT C. Thus, it is contemplated that the vaccines of the present invention can be administered once to a mammal, or repeated boost vaccinations can be administered following a sufficient delay following a first dosage.

TABLE 1
Comparison of Native (SEQ ID NO: 1) and Codon-optimized
(SEQ ID NO: 2) BoNT/C-H_{C}50 DNA and Protein (SEQ ID NO: 3) Sequences

| | Position | Sequence |
|---|---|---|
| Native | 1-36 | GAT ATA ATT AAT GAA TAT TTC AAT AAT ATT AAT GAT |
| Optimized | 1-36 | GAC ATT ATC AAC GAG TAC TTC AAT AAC ATC AAT GAC |
| Protein | 1-12 | D I I N E Y F N N I N D |
| Native | 37-72 | TCA AAA ATT TTG AGC CTA CAA AAC AGA AAA AAT ACT |
| Optimized | 37-72 | AGC AAG ATC CTG TCC CTG CAA AAT CGG AAA AAC ACT |
| Protein | 13-24 | S K I L S L Q N R K N T |
| Native | 73-108 | TTA GTG GAT ACA TCA GGA TAT AAT GCA GAA GTG AGT |
| Optimized | 73-108 | CTG GTG GAC ACC AGC GGA TAT AAC GCT GAG GTG AGC |
| Protein | 25-36 | L V D T S G Y N A E V S |
| Native | 109-144 | GAA GAA GGC GAT GTT CAG CTT AAT CCA ATA TTT CCA |
| Optimized | 109-144 | GAA GAA GGC GAT GTG CAA CTG AAC CCA ATC TTC CCC |
| Protein | 37-48 | E E G D V Q L N P I F P |
| Native | 145-180 | TTT GAC TTT AAA TTA GGT AGT TCA GGG GAG GAT AGA |
| Optimized | 145-180 | TTT GAT TTT AAG CTG GGC TCC TCC GGC GAG GAT AGG |
| Protein | 49-60 | F D F K L G S S G E D R |
| Native | 181-216 | GGT AAA GTT ATA GTA ACC CAG AAT GAA AAT ATT GTA |
| Optimized | 181-216 | GGG AAA GTC ATC GTC ACC CAG AAT GAA AAC ATC GTC |
| Protein | 61-72 | G K V I V T Q N E N I V |
| Native | 217-252 | TAT AAT TCT ATG TAT GAA AGT TTT AGC ATT AGT TTT |
| Optimized | 217-252 | TAC AAT AGC ATG TAC GAG AGC TTC AGC ATC TCC TTC |
| Protein | 73-84 | Y N S M Y E S F S I S F |
| Native | 253-288 | TGG ATT AGA ATA AAT AAA TGG GTA AGT AAT TTA CCT |
| Optimized | 253-288 | TGG ATC AGA ATT AAC AAA TGG GTC AGC AAC CTG CCA |
| Protein | 85-96 | W I R I N K W V S N L P |
| Native | 289-324 | GGA TAT ACT ATA ATT GAT AGT GTT AAA AAT AAC TCA |
| Optimized | 289-324 | GGA TAT ACC ATC ATC GAC AGC GTG AAG AAC AAC TCC |
| Protein | 97-108 | G Y T I I D S V K N N S |
| Native | 325-360 | GGT TGG AGT ATA GGT ATT ATT AGT AAT TTT TTA GTA |
| Optimized | 325-360 | GGG TGG TCC ATC GGG ATT ATC TCC AAT TTT CTG GTG |
| Protein | 109-120 | G W S I G I I S N F L V |
| Native | 361-396 | TTT ACT TTA AAA CAA AAT GAA GAT AGT GAA CAA AGT |
| Optimized | 361-396 | TTC ACT CTG AAA CAA AAC GAA GAT AGC GAA CAG AGC |
| Protein | 121-132 | F T L K Q N E D S E Q S |
| Native | 397-432 | ATA AAT TTT AGT TAT GAT ATA TCA AAT AAT GCT CCT |
| Optimized | 397-432 | ATC AAT TTC TCC TAC GAC ATT TCC AAC AAT GCA CCA |
| Protein | 133-144 | I N F S Y D I S N N A P |
| Native | 433-468 | GGA TAC AAT AAA TGG TTT TTT GTA ACT GTT ACT AAC |
| Optimized | 433-468 | GGG TAT AAC AAG TGG TTC TTT GTC ACT GTC ACC AAC |
| Protein | 145-156 | G Y N K W F F V T V T N |
| Native | 469-504 | AAT ATG ATG GGA AAT ATG AAG ATT TAT ATA AAT GGA |
| Optimized | 469-504 | AAC ATG ATG GGG AAC ATG AAG ATT TAC ATC AAC GGA |
| Protein | 157-168 | N M M G N M K I Y I N G |
| Native | 505-540 | AAA TTA ATA GAT ACT ATA AAA GTT AAA GAA CTA ACT |
| Optimized | 505-540 | AAA CTG ATT GAT ACT ATT AAG GTC AAG GAA CTC ACC |
| Protein | 169-180 | K L I D T I K V K E L T |
| Native | 541-576 | GGA ATT AAT TTT AGC AAA ACT ATA ACA TTT GAA ATA |
| Optimized | 541-576 | GGC ATT AAC TTC TCC AAG ACA ATT ACA TTT GAG ATC |
| Protein | 181-192 | G I N F S K T I T F E I |
| Native | 577-612 | AAT AAA ATT CCA GAT ACC GGT TTG ATT ACT TCA GAT |
| Optimized | 577-612 | AAT AAG ATT CCA GAC ACC GGA CTC ATT ACT AGC GAC |
| Protein | 193-204 | N K I P D T G L I T S D |
| Native | 613-648 | TCT GAT AAC ATC AAT ATG TGG ATA AGA GAT TTT TAT |
| Optimized | 613-648 | TCC GAC AAT ATC AAT ATG TGG ATT AGG GAC TTC TAC |
| Protein | 205-216 | S D N I N M W I R D F Y |
| Native | 649-684 | ATA TTT GCT AAA GAA TTA GAT GGT AAA GAT ATT AAT |
| Optimized | 649-684 | ATC TTT GCT AAA GAA CTG GAT GGC AAG GAT ATT AAC |
| Protein | 217-228 | I F A K E L D G K D I N |
| Native | 685-720 | ATA TTA TTT AAT AGC TTG CAA TAT ACT AAT GTT GTA |
| Optimized | 685-720 | ATT CTC TTC AAC TCC CTC CAA TAC ACA AAC GTC GTC |
| Protein | 229-240 | I L F N S L Q Y T N V V |
| Native | 721-756 | AAA GAT TAT TGG GGA AAT GAT TTA AGA TAT AAT AAA |
| Optimized | 721-756 | AAA GAC TAT TGG GGC AAC GAC CTG AGA TAC AAC AAA |
| Protein | 241-252 | K D Y W G N D L R Y N K |
| Native | 757-792 | GAA TAT TAT ATG GTT AAT ATA GAT TAT TTA AAT AGA |
| Optimized | 757-792 | GAG TAT TAC ATG GTC AAC ATC GAT TAC CTG AAC AGA |
| Protein | 253-264 | E Y Y M V N I D Y L N R |
| Native | 793-828 | TAT ATG TAT GCG AAC TCA CGA CAA ATT GTT TTT AAT |
| Optimized | 793-828 | TAT ATG TAC GCC AAC AGC AGG CAA ATT GTG TTC AAC |
| Protein | 265-276 | Y M Y A N S R Q I V F N |
| Native | 829-864 | ACA CGT AGA AAT AAT AAT GAC TTC AAT GAA GGA TAT |
| Optimized | 829-864 | ACA CGG AGG AAT AAC AAT GAT TTC AAC GAA GGC TAT |
| Protein | 277-288 | T R R N N N D F N E G Y |
| Native | 865-900 | AAA ATT ATA ATA AAA AGA ATC AGA GGA AAT ACA AAT |
| Optimized | 865-900 | AAG ATC ATC ATC AAA AGA ATC AGG GGA AAC ACT AAT |
| Protein | 289-300 | K I I I K R I R G N T N |
| Native | 901-936 | GAT ACT AGA GTA CGA GGA GGA GAT ATT TTA TAT TTT |
| Optimized | 901-936 | GAC ACT AGG GTC AGA GGC GGC GAC ATT CTG TAT TTT |
| Protein | 301-312 | D T R V R G G D I L Y F |
| Native | 937-972 | GAT ATG ACA ATT AAT AAC AAA GCA TAT AAT TTG TTT |
| Optimized | 937-972 | GAC ATG ACT ATC AAC AAT AAG GCC TAC AAC CTG TTT |
| Protein | 313-324 | D M T I N N K A Y N L F |
| Native | 973-1008 | ATG AAG AAT GAA ACT ATG TAT GCA GAT AAT CAT AGT |
| Optimized | 973-1008 | ATG AAA AAC GAG ACA ATG TAT GCT GAT AAC CAC AGC |
| Protein | 325-336 | M K N E T M Y A D N H S |
| Native | 1009-1044 | ACT GAA GAT ATA TAT GCT ATA GGT TTA AGA GAA CAA |
| Optimized | 1009-1044 | ACA GAA GAT ATT TAC GCA ATC GGC CTG AGG GAG CAA |
| Protein | 337-348 | T E D I Y A I G L R E Q |
| Native | 1045-1080 | ACA AAG GAT ATA AAT GAT AAT ATT ATA TTT CAA ATA |
| Optimized | 1045-1080 | ACC AAA GAC ATT AAC GAT AAT ATC ATT TTC CAG ATC |
| Protein | 349-360 | T K D I N D N I I F Q I |
| Native | 1081-1116 | CAA CCA ATG AAT AAT ACT TAT TAT TAC GCA TCT CAA |
| Optimized | 1081-1116 | CAG CCA ATG AAT AAT ACC TAC TAC TAC GCA AGC CAA |
| Protein | 361-372 | Q P M N N T Y Y Y A S Q |
| Native | 1117-1152 | ATA TTT AAA TCA AAT TTT AAT GGA GAA AAT ATT TCT |
| Optimized | 1117-1152 | ATT TTC AAG AGC AAC TTT AAC GGA GAG AAC ATC AGC |
| Protein | 373-384 | I F K S N F N G E N I S |
| Native | 1153-1188 | GGA ATA TGT TCA ATA GGT ACT TAT CGT TTT AGA CTT |
| Optimized | 1153-1188 | GGA ATC TGC AGC ATT GGG ACC TAC AGG TTT AGA CTC |
| Protein | 385-396 | G I C S I G T Y R F R L |
| Native | 1189-1224 | GGA GGT GAT TGG TAT AGA CAC AAT TAT TTG GTG CCT |
| Optimized | 1189-1224 | GGG GGA GAC TGG TAT AGA CAT AAT TAC CTC GTG CCT |
| Protein | 397-408 | G G D W Y R H N Y L V P |
| Native | 1225-1260 | ACT GTG AAG CAA GGA AAT TAT GCT TCA TTA TTA GAA |
| Optimized | 1225-1260 | ACC GTC AAG CAG GGA AAT TAT GCC AGC CTC CTC GAA |
| Protein | 409-420 | T V K Q G N Y A S L L E |
| Native | 1261-1296 | TCA ACA TCA ACT CAT TGG GGT TTT GTA CCT GTA AGT |
| Optimized | 1261-1296 | AGC ACT TCC ACC CAT TGG GGA TTT GTC CCC GTC TCC |
| Protein | 421-432 | S T S T H W G F V P V S |
| Native | 1297-1299 | GAA |
| Optimized | 1297-1302 | GAG TGA |
| Protein | 433 | E * |

TABLE 2
Comparison of Native (SEQ ID NO: 4) and Codon-optimized
(SEQ ID NO: 5) BoNT/A-H_{C}50 DNA and Protein (SEQ ID NO: 6) Sequences

| | Position | Sequence |
|---|---|---|
| Native | 1-36 | AGA TTA TTA TCT ACA TTT ACT GAA TAT ATT AAG AAT |
| Optimized | 1-36 | CGG CTC CTG TCC ACT TTC ACA GAA TAT ATC AAA AAC |
| Protein | 1-12 | R L L S T F T E Y I K N |
| Native | 37-72 | ATT ATT AAT ACT TCT ATA TTG AAT TTA AGA TAT GAA |
| Optimized | 37-72 | ATT ATC AAT ACT AGC ATC CTG AAT CTC CGG TAT GAG |
| Protein | 13-24 | I I N T S I L N L R Y E |
| Native | 73-108 | AGT AAT CAT TTA ATA GAC TTA TCT AGG TAT GCA TCA |
| Optimized | 73-108 | AGC AAC CAC CTG ATT GAC CTG AGC AGG TAC GCA AGC |
| Protein | 25-36 | S N H L I D L S R Y A S |
| Native | 109-144 | AAA ATA AAT ATT GGT AGT AAA GTA AAT TTT GAT CCA |
| Optimized | 109-144 | AAA ATC AAC ATC GGC TCC AAG GTG AAC TTT GAC CCC |
| Protein | 37-48 | K I N I G S K V N F D P |
| Native | 145-180 | ATA GAT AAA AAT CAA ATT CAA TTA TTT AAT TTA GAA |
| Optimized | 145-180 | ATC GAT AAG AAC CAA ATT CAA CTC TTT AAT CTC GAA |
| Protein | 49-60 | I D K N Q I Q L F N L E |
| Native | 181-216 | AGT AGT AAA ATT GAG GTA ATT TTA AAA AAT GCT ATT |
| Optimized | 181-216 | TCC AGC AAG ATT GAG GTC ATT CTG AAA AAC GCT ATC |
| Protein | 61-72 | S S K I E V I L K N A I |
| Native | 217-252 | GTA TAT AAT AGT ATG TAT GAA AAT TTT AGT ACT AGC |
| Optimized | 217-252 | GTG TAC AAC TCC ATG TAC GAG AAC TTT TCC ACC AGC |
| Protein | 73-84 | V Y N S M Y E N F S T S |
| Native | 253-288 | TTT TGG ATA AGA ATT CCT AAG TAT TTT AAC AGT ATA |
| Optimized | 253-288 | TTC TGG ATT AGG ATC CCA AAA TAC TTC AAT AGC ATT |
| Protein | 85-96 | F W I R I P K Y F N S I |
| Native | 289-324 | AGT CTA AAT AAT GAA TAT ACA ATA ATA AAT TGT ATG |
| Optimized | 289-324 | TCC CTC AAT AAC GAG TAT ACC ATC ATC AAT TGT ATG |
| Protein | 97-108 | S L N N E Y T I I N C M |
| Native | 325-360 | GAA AAT AAT TCA GGA TGG AAA GTA TCA CTT AAT TAT |
| Optimized | 325-360 | GAA AAC AAT AGC GGC TGG AAG GTG TCC CTG AAT TAC |
| Protein | 109-120 | E N N S G W K V S L N Y |
| Native | 361-396 | GGT GAA ATA ATC TGG ACT TTA CAG GAT ACT CAG GAA |
| Optimized | 361-396 | GGA GAG ATC ATC TGG ACT CTG CAA GAC ACC CAG GAG |
| Protein | 121-132 | G E I I W T L Q D T Q E |
| Native | 397-432 | ATA AAA CAA AGA GTA GTT TTT AAA TAC AGT CAA ATG |
| Optimized | 397-432 | ATC AAA CAG AGA GTC GTG TTC AAA TAC TCC CAA ATG |
| Protein | 133-144 | I K Q R V V F K Y S Q M |
| Native | 433-468 | ATT AAT ATA TCA GAT TAT ATA AAC AGA TGG ATT TTT |
| Optimized | 433-468 | ATT AAC ATT AGC GAC TAC ATC AAC AGA TGG ATC TTC |
| Protein | 145-156 | I N I S D Y I N R W I F |
| Native | 469-504 | GTA ACT ATC ACT AAT AAT AGA TTA AAT AAC TCT AAA |
| Optimized | 469-504 | GTG ACA ATT ACA AAC AAC AGG CTG AAT AAC TCC AAG |
| Protein | 157-168 | V T I T N N R L N N S K |
| Native | 505-540 | ATT TAT ATA AAT GGA AGA TTA ATA GAT CAA AAA CCA |
| Optimized | 505-540 | ATT TAC ATT AAC GGC AGG CTC ATC GAT CAG AAG CCT |
| Protein | 169-180 | I Y I N G R L I D Q K P |
| Native | 541-576 | ATT TCA AAT TTA GGT AAT ATT CAT GCT AGT AAT AAT |
| Optimized | 541-576 | ATT AGC AAC CTC GGC AAT ATT CAT GCC AGC AAT AAC |
| Protein | 181-192 | I S N L G N I H A S N N |
| Native | 577-612 | ATA ATG TTT AAA TTA GAT GGT TGT AGA GAT ACA CAT |
| Optimized | 577-612 | ATC ATG TTT AAG CTC GAC GGC TGT AGG GAT ACC CAC |
| Protein | 193-204 | I M F K L D G C R D T H |
| Native | 613-648 | AGA TAT ATT TGG ATA AAA TAT TTT AAT CTT TTT GAT |
| Optimized | 613-648 | AGG TAC ATT TGG ATC AAG TAC TTT AAC CTC TTT GAC |
| Protein | 205-216 | R Y I W I K Y F N L F D |
| Native | 649-684 | AAG GAA TTA AAT GAA AAA GAA ATC AAA GAT TTA TAT |
| Optimized | 649-684 | AAG GAA CTG AAC GAA AAA GAA ATC AAA GAT CTC TAC |
| Protein | 217-228 | K E L N E K E I K D L Y |
| Native | 685-720 | GAT AAT CAA TCA AAT TCA GGT ATT TTA AAA GAC TTT |
| Optimized | 685-720 | GAT AAC CAA AGC AAC TCC GGA ATT CTC AAA GAC TTT |
| Protein | 229-240 | D N Q S N S G I L K D F |
| Native | 721-756 | TGG GGT GAT TAT TTA CAA TAT GAT AAA CCA TAC TAT |
| Optimized | 721-756 | TGG GGC GAT TAC CTG CAA TAC GAC AAG CCA TAC TAC |
| Protein | 241-252 | W G D Y L Q Y D K P Y Y |
| Native | 757-792 | ATG TTA AAT TTA TAT GAT CAA AAT AAA TAT GTC GAT |
| Optimized | 757-792 | ATG CTC AAC CTG TAT GAC CCT AAC AAA TAC GTG GAT |
| Protein | 253-264 | M L N L Y D P N K Y V D |
| Native | 793-828 | GTA AAT AAT GTA GGT ATT AGA GGT TAT ATG TAT CTT |
| Optimized | 793-828 | GTC AAT AAT GTG GGC ATC CGG GGG TAT ATG TAT CTC |
| Protein | 265-276 | V N N V G I R G Y M Y L |
| Native | 829-864 | AAA GGG CCT AGA GGT AGC GTA ATG ACT ACA AAC ATT |
| Optimized | 829-864 | AAG GGA CCT CGG GGA AGC GTG ATG ACC ACA AAT ATC |
| Protein | 277-288 | K G P R G S V M T T N I |
| Native | 865-900 | TAT TTA AAT TCA AGT TTG TAT AGG GGG ACA AAA TTT |
| Optimized | 865-900 | TAC CTG AAC AGC TCC CTG TAT AGA GGC ACT AAA TTC |
| Protein | 289-300 | Y L N S S L Y R G T K F |
| Native | 901-936 | ATT ATA AAA AAA TAT GCT TCT GGA AAT AAA GAT AAT |
| Optimized | 901-936 | ATC ATT AAG AAG TAT GCT AGC GGA AAC AAG GAT AAC |
| Protein | 301-312 | I I K K Y A S G N K D N |
| Native | 937-972 | ATT GTT AGA AAT AAT GAT CGT GTA TAT ATT AAT GTA |
| Optimized | 937-972 | ATT GTG CGG AAC AAC GAC AGA GTC TAT ATT AAC GTC |
| Protein | 313-324 | I V R N N D R V Y I N V |
| Native | 973-1008 | GTA GTT AAA AAT AAA GAA TAT AGG TTA GCT ACT AAT |
| Optimized | 973-1008 | GTC GTC AAG AAC AAA GAG TAC AGA CTG GCA ACA AAC |
| Protein | 325-336 | V V K N K E Y R L A T N |
| Native | 1009-1044 | GCA TCA CAG GCA GGC GTA GAA AAA ATA CTA AGT GCA |
| Optimized | 1009-1044 | GCA TCC CAG GCT GGA GTC GAA AAG ATT CTC AGC GCT |
| Protein | 337-348 | A S Q A G V E K I L S A |
| Native | 1045-1080 | TTA GAA ATA CCT GAT GTA GGA AAT CTA AGT CAA GTA |
| Optimized | 1045-1080 | CTG GAA ATT CCT GAC GTG GGC AAT CTC AGC CAG GTC |
| Protein | 349-360 | L E I P D V G N L S Q V |
| Native | 1081-1116 | GTA GTA ATG AAG TCA AAA AAT GAT CAA GGA ATA ACA |
| Optimized | 1081-1116 | GTG GTG ATG AAA AGC AAG AAC GAT CAG GGC ATC ACT |
| Protein | 361-372 | V V M K S K N D Q G I T |
| Native | 1117-1152 | AAT AAA TGC AAA ATG AAT TTA CAA GAT AAT AAT GGG |
| Optimized | 1117-1152 | AAT AAG TGT AAG ATG AAC CTC CAG GAC AAC AAC GGG |
| Protein | 373-384 | N K C K M N L Q D N N G |
| Native | 1153-1188 | AAT GAT ATA GGC TTT ATA GGA TTT CAT CAG TTT AAT |
| Optimized | 1153-1188 | AAT GAT ATC GGA TTC ATT GGG TTT CAC CAG TTT AAC |
| Protein | 385-396 | N D I G F I G F H Q F N |
| Native | 1189-1224 | AAT ATA GCT AAA CTA GTA GCA AGT AAT TGG TAT AAT |
| Optimized | 1189-1224 | AAC ATT GCT AAA CTC GTC GCC TCC AAC TGG TAC AAT |
| Protein | 397-408 | N I A K L V A S N W Y N |
| Native | 1225-1260 | AGA CAA ATA GAA AGA TCT AGT AGG ACT TTG GGT TGC |
| Optimized | 1225-1260 | AGG CAG ATC GAG AGA AGC AGC AGA ACA CTC GGG TGT |
| Protein | 409-420 | R Q I E R S S R T L G C |
| Native | 1261-1296 | TCA TGG GAA TTT ATT CCT GTA GAT GAT GGA TGG GGA |
| Optimized | 1261-1296 | AGC TGG GAG TTC ATC CCC GTC GAT GAC GGG TGG GGA |
| Protein | 421-432 | S W E F I P V D D G W G |
| Native | 1297-1311 | GAA AGG CCA CTG TAA |
| Optimized | 1297-1311 | GAA CGG CCT CTC TGA |
| Protein | 433-436 | E R P L * |

A further aspect of the present invention relates an isolated antibody raised against a chimeric protein of the present invention, or antibody fragment thereof, which antibody or antibody fragment is capable of specifically binding and neutralizing a Clostridium botulinum neurotoxin.

The antibodies of the present invention can be polyclonal antibodies or monoclonal antibodies, although monoclonal antibodies are preferred because of their specificity. The antibody can also be a polyclonal preparation rendered monospecific.

Various methods of producing antibodies with a known antigen are well-known to those ordinarily skilled in the art (ANTIBODIES: A LABORATORY MANUAL (Harlow & Lane eds., 1988), which is hereby incorporated by reference in its entirety). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In particular, monoclonal antibody production may be effected by techniques which are well-known in the art. Basically, the process involves first obtaining immune cells (lymphocytes) from the spleen of a mammal (e.g., mouse) which has been previously immunized with the chimeric protein either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler & Milstein, “Continuous Cultures of Fused Cells Secreting Antibody of Predefined Specificity,” Nature, 256:495-497 (1975), which is hereby incorporated by reference in its entirety.

Mammalian lymphocytes are immunized by in vivo immunization of the animal (e.g., a mouse) with the chimeric protein or DNA vaccine of the present invention. Following a sufficient number of immunizations (i.e., one or more), the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (see Milstein & Kohler, “Derivation of Antibody-producing Tissue Culture and Tumor Lines by Cell Fusion,” Eur. J. Immunol. 6:511-519 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the chimeric protein or DNA vaccine of the present invention subcutaneously to New Zealand white rabbits which have first been bled to obtain pre-immune serum. The antigens can be administered at a total volume of 100 μl per site at multiple sites. Each injected material may contain synthetic surfactant adjuvant pluronic polyols, or pulverized acrylamide gel containing the purified chimeric protein or DNA vaccine. The rabbits are then bled two weeks after the first injection and periodically boosted with the same antigen three times every six weeks. Boosting may not be required with the DNA vaccine. A sample of serum is then collected 10 days after each boost. Polyclonal antibodies are then recovered from the serum by affinity chromatography using the corresponding chimeric protein to capture the antibody. Ultimately, the rabbits are euthanized with pentobarbital 150 mg/Kg IV. This and other procedures for raising polyclonal antibodies are disclosed in ANTIBODIES: A LABORATORY MANUAL (Harlow & Lane eds., 1988), which is hereby incorporated by reference in its entirety.

In addition to utilizing whole antibodies, the present invention also encompasses use of binding portions of such antibodies. Such binding portions include Fab fragments, F(ab)₂ fragments, Fab′ fragments, F(ab)₂ fragments, Fd fragments, Fd′ fragments, Fv fragments, and minibodies, e.g., 61-residue subdomains of the antibody heavy-chain variable domain (Pessi et al., “A Designed Metal-binding Protein with a Novel Fold,” Nature 362:367-369 (1993), which is hereby incorporated by reference in its entirety). Domain antibodies (dAbs) are also suitable for the methods of the present invention (Holt et al., “Domain Antibodies: Proteins for Therapy,” Trends Biotechnol. 21:484-90 (2003), which is hereby incorporated by reference in its entirety). These antibody fragments can be made by conventional procedures, such as proteolytic fragmentation procedures, as described in J. Goding, MONOCLONAL ANTIBODIES: PRINCIPLES AND PRACTICE 98-118 (1984), which is hereby incorporated by reference in its entirety.

The antibodies may be from humans, or from animals other than humans, preferably mammals, such as rat, mouse, guinea pig, rabbit, goat, sheep, and pig, or avian species such as chicken. Preferred are mouse monoclonal antibodies and antigen-binding fragments or portions thereof. In addition, chimeric antibodies and hybrid antibodies are embraced by the present invention. Techniques for the production of chimeric antibodies are described in, e.g., Morrison et al., “Chimeric Human Antibody Molecules: Mouse Antigen-binding Domains with Human Constant Region Domains,” Proc. Nat'l Acad. Sci. USA 81:6851-5 (1984), Neuberger et al., “Recombinant Antibodies Possessing Novel Effector Functions,” Nature 312:604-8 (1984), and Takeda et al., “Construction of Chimaeric Processed Immunoglobulin Genes Containing Mouse Variable and Human Constant Region Sequences,” Nature 314:452-4 (1985), each of which is hereby incorporated by reference in its entirety. For human therapeutic purposes, humanized antibodies or fragments are preferred.

Further, single chain antibodies are also suitable for the present invention (e.g., U.S. Pat. Nos. 5,476,786 to Huston and 5,132,405 to Huston & Oppermann; Huston et al., “Protein Engineering of Antibody Binding Sites: Recovery of Specific Activity in an Anti-digoxin Single-chain Fv Analogue Produced in Escherichia coli,” Proc. Nat'l Acad. Sci. USA 85:5879-83 (1988); U.S. Pat. No. 4,946,778 to Ladner et al.; Bird et al., “Single-chain Antigen-binding Proteins,” Science 242:423-6 (1988); Ward et al., “Binding Activities of a Repertoire of Single Immunoglobulin Variable Domains Secreted from Escherichia coli,” Nature 341:544-6 (1989), each of which is hereby incorporated by reference in its entirety). Single chain antibodies are formed by linking the heavy and light immunoglobulin chain fragments of the Fv region via an amino acid bridge, resulting in a single chain polypeptide. Univalent antibodies are also embraced by the present invention.

A pharmaceutical composition comprising the antibodies or antibody fragments of the present invention can be administered to an individual to provide passive immunity against a botulism neurotoxin. The pharmaceutical composition can include antibodies or antibody fragments against a single botulism neurotoxin, or the composition can contain antibodies or antibody fragments against any two or more botulism neurotoxin (e.g., neurotoxin A, neurotoxin B, neurotoxin C, neurotoxin D, neurotoxin E, neurotoxin F, and neurotoxin G).

The antibodies or antibody fragments can be administered to a patient exposed to Clostridium botulinum to afford passive immunity against a botulism neurotoxin. Thus, a further aspect of the present invention relates to treatment of a Clostridium botulinum infection by administering to a patient the antibodies or antibody fragments (or composition containing the same) under conditions effective to neutralize the botulism neurotoxin. Administration can be carried out by any suitable means, but preferably parenterally, subcutaneously, intravenously, intramuscularly, intraperitoneally, by intracavitary or intravesical instillation, intraarterially, intralesionally, by application to mucous membranes, or directly to a site of infection. The amount of antiserum administered should be sufficient to neutralize the neurotoxin, i.e., in excess. This method of treatment is typically carried out in combination with other therapeutic agents, e.g., antibiotics, sufficient to destroy the Clostridium botulinum population.

### EXAMPLES

The following examples are provided to illustrate embodiments of the present invention but they are by no means intended to limit its scope.

### Materials and Methods

### Animals

Six to eight-week old, female Balb/c mice, were purchased from Taconic Farms (Hudson, N.Y.), and housed in the animal facility of University of Rochester (4 animals per cage). They were maintained in a controlled environment (22±2° C.; 12 h light/12 h dark cycles) in accordance with the U.S. Public Health Service “Guide for the Care and Use of Laboratory Animals.” The animals were provided Laboratory Rodent Diet 5001 with ad libitum access to food and water. The research was conducted in compliance with the Animal Welfare Act and other federal and state statutes and regulations relating to animals and experiments involving animals and adheres to principles stated in the Guide for the Care and Use of Laboratory Animals, NRC Publication, 1996 edition.

### Saliva, Nasal Wash, and Vagina Wash Specimens

For these experiments, 16 mice were divided into 2 test groups and 2 control groups, 8 mice per group. The animals were vaccinated with 2×10⁷ pfu/mouse of Ad/opt-BoNT/C—H_{C}50 in the test groups and same amount of Ad/Null in the control groups at week 0, Saliva, nasal wash, and vaginal wash samples were collected at week 2 in one test group and one control group, and week 4 in one test group and one control group for each time point. The mice were anesthetized by i.p. injection with 2 mg of ketamine HCl (Bedford Laboratories, Bedford, Ohio) plus 0.2 mg of xylazine (Butler Company, Columbus, Ohio) in 100 μl. Vaginal washes were collected by flushing the vagina with 100 μl PBS by repeated aspiration using a pipette with an animal feeding needle (with a ball head) until turbid (Singh et al., “Mucosal Immunization with Recombinant MOMP Genetically Linked with Modified Cholera Toxin Confers Protection Against Chlamydia Trachomatis Infection,” Vaccine 24:1213-1224 (2006), which is hereby incorporated by reference in its entirety). The saliva samples were collected using a 200 μl pipette fitted with a plastic tip, after i.p. injection of carbachol (Sigma Chemical Co., St. Louis, Mo.; 10 μg in 0.1 ml) to stimulate salivation as described previously (Russell et al., “Distribution, Persistence, and Recall of Serum and Salivary Antibody Responses to Peroral Immunization with Protein Antigen I/II of Streptococcus mutans Coupled to the Cholera Toxin B Subunit,” Infect Immun 59:4061-4070 (1991); Zeng et al., “Protection Against Anthrax by needle-Free Mucosal Immunization with Human Anthrax Vaccine,” Vaccine 25:3558-3594 (2007), each of which is hereby incorporated by reference in its entirety). The mice were then incised ventrally along the median line from the xiphoid process to the chin, the heads were removed, and the lower jaws were excised. A hypodermic needle was inserted into the posterior opening of the nasopharynx and 200 μl of PBS was injected repeatedly to collect the nasal wash samples (Watanabe et al., “Characterization of Protective Immune Responses Induced by Nasal Influenza Vaccine Containing Mutant Cholera Toxin as a Safe Adjuvant (CT112K),” Vaccine 20:3443-3455 (2002), which is hereby incorporated by reference in its entirety).

Challenge with Botulinum Neurotoxin

All animals were challenged by i.p injection with 10²-10⁵×MLD₅₀ of C. botulinum neurotoxin BoNT/C (Metabiolgics Inc, Madison, Wis.) per mouse as specified in each experiment. The challenged animals were monitored for 7 days. They were observed every 4 h for the first two days and twice a day thereafter. The number of deaths for each group was recorded as the endpoint (Arimitsu et al., “Vaccination With Recombinant Whole Heavy Chain Fragments of Clostridium Botulinum Type C and D Neurotoxins.” Clin Diagn Lab Immunol 11(3):496-502 (2004), which is hereby incorporated by reference in its entirety).

### ELISA for Determination of Antibody Concentration

Anti-BoNT/C—H_{C}50 IgG, IgG1, IgG2a, and IgA antibody concentrations in serum, saliva, nasal wash and/or vaginal wash samples were determined using an ELISA Quantization kit (Bethel Lab. Inc., Montgomery Tex.) with a modified procedure. Briefly, 96-well flat-bottom immuno plates (Nagle Nunc International, Rochester, N.Y.) were coated with 0.5 μg/well of either His-tagged BoNT/C—H_{C}50 recombinant protein produced in Escherichia coli or capture antibodies (goat anti-mouse IgG-, or IgG1-, IgG2a-, or IgA-affinity purified, Bethel Lab, Montgomery, Tex., for standard curve) in 100 μl coating buffer (0.05M carbonate-bicarbonate buffer, pH 9.6) at 4° C. overnight. The plates were washed 5 times with washing buffer (0.05% Tween 20 in PBS) and nonspecific binding sites were blocked with 200 μl PBS (pH 7.4) containing 1% bovine serum albumin (BSA) for 1 h at room temperature. After five washes, 100 μl serial dilutions of reference serum containing given amounts of mouse antibodies (for standard curve) or 1:100 dilutions of mouse serum samples in PBS (pH 7.4) containing 0.05% Tween 20 and 1% BSA were added. After 2 h further incubation at 37° C., the plates were washed with washing buffer 5 times and incubated with 100 μl/well of 1:10,000 dilution of goat anti-mouse IgG, IgG1, IgG2a, or IgA conjugated to alkaline phosphatase for 1 h at room temperature. Unbound antibodies were removed by washing 5 times with washing buffer, and the bound antibody was detected after incubation with p-nitrophenylphosphate phosphatase substrate system (KPL, Gaithersburg, Md.) for 30 min. The color reaction was terminated by adding 100 μA 0.5 M EDTA and the absorbance values were obtained using a Dynatech MR4000 model microplate reader at 405 nm. A standard curve for antibody quantitation was generated in parallel to allow antibody concentration calculations, as previously described (see Zeng et al., “Protection Against Anthrax by Needle-Free Mucosal Immunization With Human Anthrax Vaccine,” Vaccine 25(18):3558-94 (2007), which is hereby incorporated by reference in its entirety).

### Anti-Adenovirus Neutralizing Antibody Titer Assay

Anti-adenovirus neutralizing antibodies were determined according to previously described methods with some modifications (Zabner et al., “Repeat Administration of an Adenovirus Vector Encoding Cystic Fibrosis Transmembrane Conductance Regulator to the Nasal Epithelium of Patients With Cystic Fibrosis.” J Clin Invest 97(6):1504-11 (1996); Harvey et al., “Variability of Human Systemic Humoral Immune Responses to Adenovirus Gene Transfer Vectors Administered to Different Organs,” J Virol 73(8):6729-42 (1999); Hashimoto et al., “Induction of Protective Immunity to Anthrax Lethal Toxin With a Nonhuman Primate Adenovirus-Based Vaccine in the Presence of Preexisting Anti-Human Adenovirus Immunity,” Infect Immun 73(10):6885-91 (2005), each of which is hereby incorporated by reference in its entirety). Briefly, AD293 cells (Stratagene, CA) were seeded in 96-well plates at a density of 10⁴ cells/well in 200 μl of Eagle's minimum essential medium (EMEM) containing 10% FBS, 2 mM glutamine, 50 U of penicillin per ml, and 50 μg of streptomycin per ml and incubated at 37° C., 5% CO₂ overnight. Mouse serum samples were heat-inactivated at 55° C. for 45 min and then serially twofold diluted in MEME containing 2% FBS in a new 96-well culture plate. Approximately 10⁴ pfu of wild-type human adenovirus Ad5 in 50 μl of MEME containing 2% FBS was mixed with 50 μA of diluted serum samples. After incubation for 1 h at 37° C. to allow neutralization to occur, 100 μA of virus-serum mixture was subsequently added to AD293 cells, and incubated for 2 h at 37° C. After incubation, the virus-serum medium was replaced with 200 μA of EMEM containing 10% FBS. The cells were incubated at 37° C. until the negative wells exhibited 90% cytopathic effect. The neutralizing antibody titer was determined to be the highest dilution wells showing <50% cytopathic effect as previously described (Zabner et al., “Repeat Administration of an Adenovirus Vector Encoding Cystic Fibrosis Transmembrane Conductance Regulator to the Nasal Epithelium of Patients With Cystic Fibrosis,” J Clin Invest 97(6):1504-11 (1996); Harvey et al., “Variability of Human Systemic Humoral Immune Responses to Adenovirus Gene Transfer Vectors Administered to Different Organs,” J Virol 73(8):6729-42 (1999); Hashimoto et al., “Induction of Protective Immunity to Anthrax Lethal Toxin With a Nonhuman Primate Adenovirus-Based Vaccine in the Presence of Preexisting Anti-Human Adenovirus Immunity,” Infect Immun 73(10):6885-91 (2005), each of which is hereby incorporated by reference in its entirety).

### Statistical Analysis

Serum and mucosal antibody concentrations among different groups at different time points were compared and analyzed using the LSD test and ANOVA/MANOVA with STATISTICA™ 7.1 software (StatSoft, Tulsa, Okla.). In comparing groups, those with P-values <0.05 and <0.01 were considered to be significant and very significant, respectively.

### Example 1

### Construction of Nucleic Acid Molecule Encoding Codon-Optimized H_{C}50 of BoNT/C Chimeric Protein, and Insertion in Adeno-viral Vector

Replication-incompetent recombinant adenoviral vectors were constructed using the AdEasy™ System (Stratagene, La Jolla, Calif.) (He et al., “A Simplified System for Generating Recombinant Adenoviruses,” Proc Natl Acad Sci USA 95(5):2509-14 (1998); Zeng et al., “AdEasy System Made Easier by Selecting the Viral Backbone Plasmid Preceding Homologous Recombination,” Biotechniques 31(2):260-2 (2001); which are hereby incorporated by reference in their entirety). The adenoviral vector is derived from human adenovirus serotype 5 rendered replication-incompetent by the deletion of the E1 and E3 regions.

To construct the Ad/opt-BoNT/C—H_{C}50, the nucleotides encoding the 50-kDa C-terminal fragment of heavy chain of botulinum neurotoxin type C1 (Kimura et al., “The Complete Nucleotide Sequence of the Gene Coding for Botulinum Type C1 Toxin in the C-ST Phage Genome,” Biochem Biophys Res Commun 171(3):1304-11 (1990), which is hereby incorporated by reference in their entirety) was optimized with human codon preference by the DNAworks program (Hoover et al., “DNAWorks: An Automated Method for Designing Oligonucleotides for PCR-Based Gene Synthesis,” Nucleic Acids Res 30(10):e43 (2002), which is hereby incorporated by reference in its entirety). The encoded 50-kDa C-terminal fragment of heavy chain of botulinum neurotoxin type C1 corresponds to amino acids 849-1291 of the BoNT/C recited in Genbank Accession No. D90210, which is hereby incorporated by reference in its entirety. The codon-optimized nucleotide sequence is shown in Table 1, supra.

The 50-kDa fragment of BoNT/C was prepared as an in-frame gene fusion with a 25 residue signal peptide of human tissue plasminogen activator (PLAT) plus two serine residues followed with the codon-optimized BoNT/C—H_{C}50. The nucleotide sequence encoding amino acids 1-25 of PLAT (MDAMKRGLCCVLLLCGAVFVSPSQE, SEQ ID NO: 7) was obtained from GenBank Accession No. BC002795, which is hereby incorporated by reference in its entirety. The nucleotide sequence corresponding to the PLAT secretion signal (with Ser-Ser linker) corresponds to SEQ ID NO: 8 as follows: atggatgcaatgaagagagggctctgctgtgtgctgctgctgtgtggagcagtcttcgtttcgcccagccaggaaagcagc. The chimeric open reading frame was then synthesized by a PCR-based method (Gao et al., “Thermodynamically Balanced Inside-Out (TBIO) PCR-Based Gene Synthesis: A Novel Method of Primer Design for High-Fidelity Assembly of Longer Gene Sequences,” Nucleic Acids Res 31(22):e143 (2003), which is hereby incorporated by reference in its entirety).

The synthesized DNA was subsequently cloned into a shuttle vector pShuttle-CMV (Stratagene, La Jolla, Calif.) at its SalI site. The DNA sequence of the synthesized gene was further confirmed by DNA sequencing analysis. The adenoviral vector was then constructed according to the standard procedures as described previously (He et al., “A Simplified System for Generating Recombinant Adenoviruses,” Proc Natl Acad Sci USA 95(5):2509-14 (1998); Zeng et al., “AdEasy System Made Easier by Selecting the Viral Backbone Plasmid Preceding Homologous Recombination,” Biotechniques 31(2):260-2 (2001), each of which is hereby incorporated by reference in its entirety). In the adenovirus, the transgene expression is under control of human cytomegalovirus (CMV) immediate early promoter/enhancer and then followed with a simian virus 40 (SV40) stop/polyadenylation signal (He et al., “A Simplified System for Generating Recombinant Adenoviruses,” Proc Natl Acad Sci USA 95(5):2509-14 (1998), which is hereby incorporated by reference in its entirety). Similarly, the Ad/Null vector without transgene was also constructed. Adenoviruses isolated from single plaques were then produced in AD293 cells (Stratagene, La Jolla, Calif.) and purified by CsCl gradient purification and dialyzed with adenovirus storage buffer containing 10 mM Tris pH 7.5, 135 mM NaCl, 5 mM KCl, 1 mM MgCl₂. The purified adenoviruses were stored in 1.0 M sucrose in a −80° C. freezer until use and their titers (pfu) were determined by plaque assay before vaccinating animals.

### Example 2

### Antibody Response to H_{C}50 of BoNT/C after Intramuscular Vaccination

Mice were allotted into five groups (8 mice/group). They were injected i.m. into the hind-leg quadriceps with different doses of Ad/opt-BoNT/C—H_{C}50 vector prepared in Example 1 (10⁵, 10⁶, or 2×10⁷ pfu/mouse), Ad/Null (2×10⁷ pfu/mouse), and the Botulinum Toxoid Adsorbed Pentavalent (ABCDE) (0.05 ml/mouse), an IND vaccine which was produced by the Michigan Department of Public Health. Animals were inoculated once in week 0. Animal sera were obtained by retro-orbital bleeding every 2 weeks (in week 0, 2, 4, and 6) and stored at −20° C. until further assays.

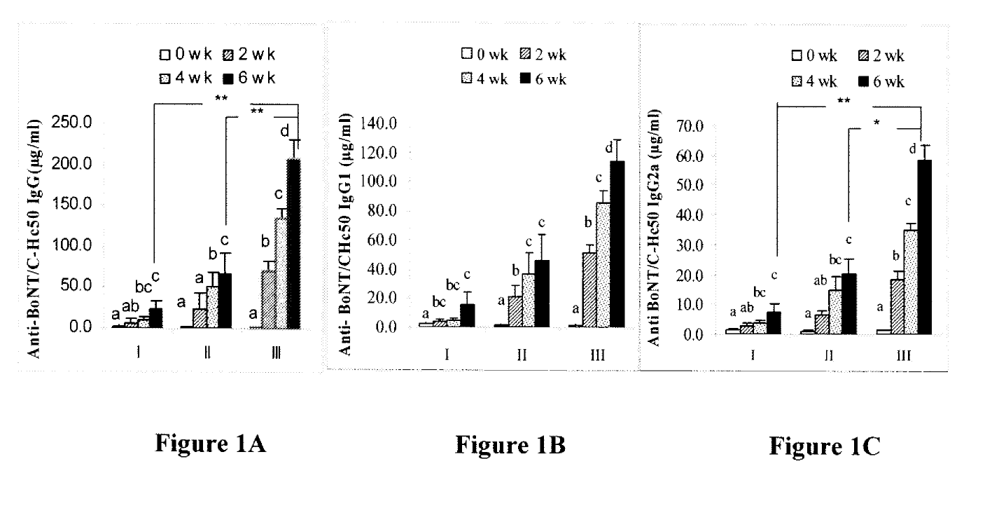

Antibody responses in animal sera were measured by quantitative ELISA. FIGS. 1A-C show BoNT/C—H_{C}50-specific antibody responses in sera 2, 4, and 6 weeks after vaccination. The data indicate that the lowest vaccine dosage 10⁵ pfu tested was sufficient to elicit significant IgG1 and IgG2a antibody responses in Week 6 compared with the control group injected with Ad/Null (an Ad5 vector with no transgene) (P values <0.05). The rise in antigen-specific IgG1 (FIG. 1B) and IgG2a (FIG. 1C) after vaccination suggested that both Th2 and Th1 immune responses were elicited. More specifically, the IgG antibody response could be characterized as a predominant Th2 response (values of IgG2a/IgG1<1.0). In addition, serum antibody responses were clearly vaccine-dose dependent.

### Example 3

### In vitro Neutralization of Botulinum Neurotoxin

Neutralizing antibody titers to BoNT/C were measured by the ability of sera to neutralize the neurotoxin in vitro in combination with the mouse lethality assay. 200 μl of pooled sera from 8 mice 6 weeks after vaccination with 2×10⁷ pfu of Ad/opt-BoNT/C—H_{C}50 vector or with Ad/Null (both obtained from inoculated mice of Example 2). The sera were initially diluted 1:4, and then diluted in twofold series (1:4 to 1:1052) in DPBS (Dulbecco's PBS). 400×MLD₅₀ BoNT/C in 200 μl of DPBS was added into each dilution. After incubation at room temperature for 1 h, the anti-serum and the BoNT/C mixture was injected i.p. into mice, 100 μl (corresponding to 100×MLD₅₀ of BoNT/C before neutralization) per mouse, 4 mice were tested for each dilution.

The mice were monitored for 4 days, and the number of deaths at each sample dilution was recorded. If the toxin was neutralized, the mice were protected from the challenge with neutralized toxin. The detection limit for this assay was 0.04 IU/ml due to the limited amount of serum available. Neutralizing antibody titers were defined as the maximum number of IU of antitoxin per ml of serum, resulting in 100% survival after challenge. One IU of botulinum neurotoxin antitoxin neutralized 10,000×MLD₅₀ neurotoxin (Byrne et al., “Purification, Potency, and Efficacy of the Botulinum Neurotoxin Type A Binding Domain from Pichia pastoris as a Recombinant Vaccine Candidate.” Infect Immun 66(10):4817-22 (1998); Nowakowski et al., “Potent Neutralization of Botulinum Neurotoxin by Recombinant Oligoclonal Antibody,” Proc Natl Acad Sci USA 99:11346-11350 (2002), each of which is hereby incorporated by reference in its entirety).

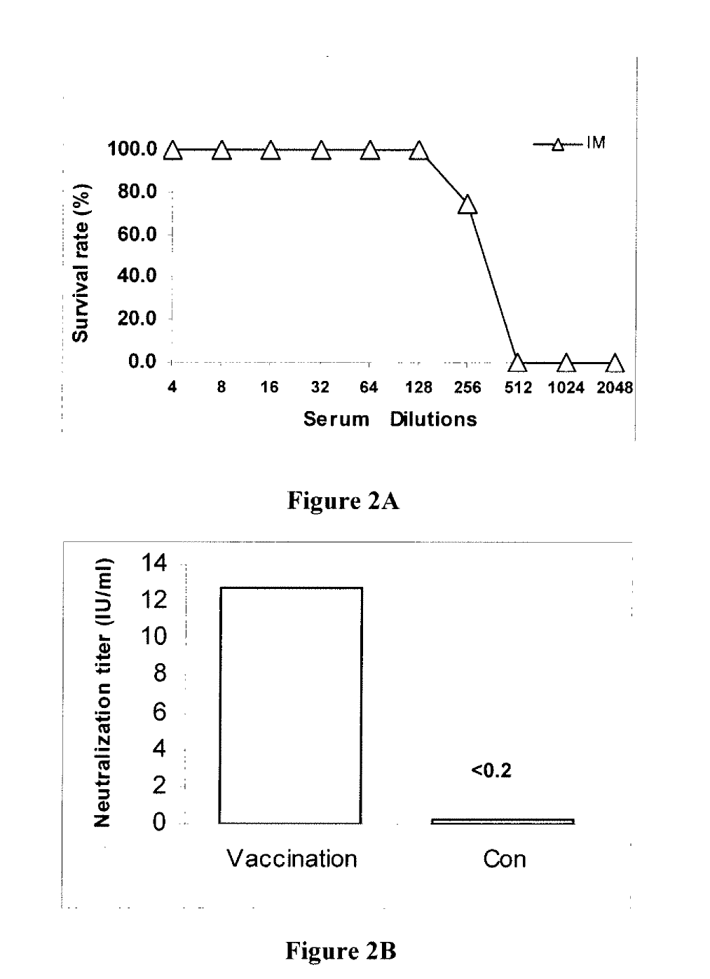

As shown in FIG. 2A, up to 128-fold diluted sera from animals 6 weeks after a single injection of 2×10⁷ pfu of Ad/opt-BoNT/C—H_{C}50 completely neutralized 100×MLD₅₀ BoNT/C under experimental conditions and resulted in a 100% survival rate after administration of the neutralized toxin in mice. This translated to a 13 IU/ml anti-BoNT/C neutralization titer (FIG. 2B). These data indicate that parental inoculation with the adenoviral vector could elicit functional antibody responses that neutralized active botulinum neurotoxin.

### Example 4

### Protective Immunity Elicited by the Adenovirus-Vectored Vaccine

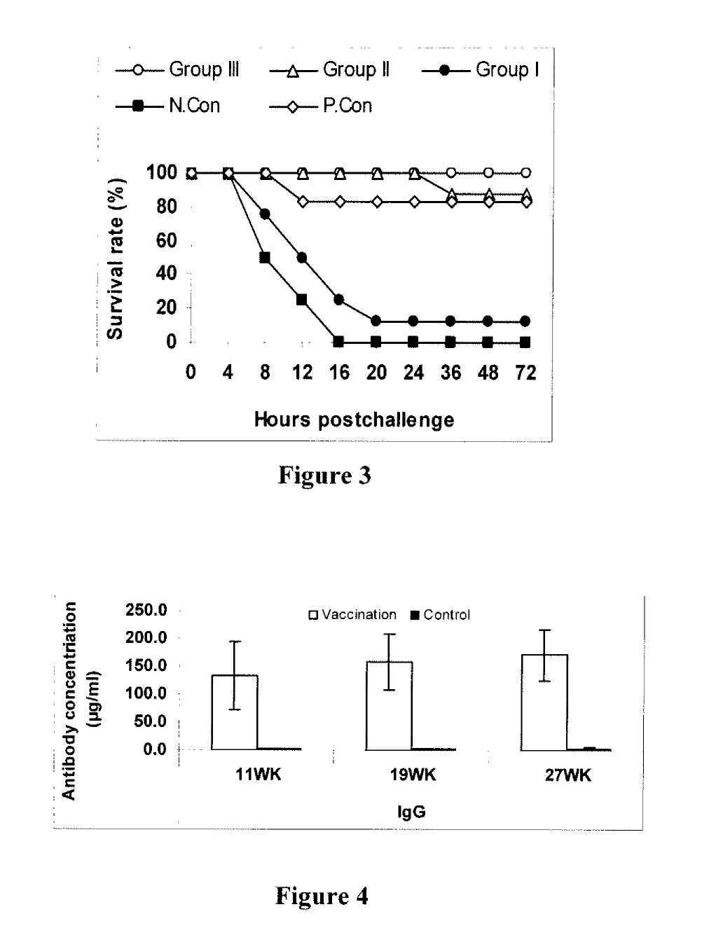

To explore whether the H_{C}50-based adenovirus-vectored vaccine protect against botulinum neurotoxin intoxication, the vaccinated mice (from Example 2) were intraperitoneally challenged (i.p.) with 100 50% mouse lethal dose (MLD₅₀) units of active BoNT/C 7 weeks after injection (i.e., one week after last bleeding). The results from the challenge experiments showed that vaccination with 10⁵ pfu of Ad/opt-BoNT/C—H_{C}50 could protect 12.5% of the animals against the toxin challenge (FIG. 3). However, the protection rates rose to 75% and 100% when the vaccine dose was increased to 10⁶ and 2×10⁷ pfu, respectively. This indicates that vaccine dose-dependent protective immunity was achieved after vaccination.

A separate experiment was also performed to determine whether a single dose of Ad/opt-BoNT/C—H_{C}50 could provide long-term immunity against botulinum neurotoxin. Mice were allotted into three experimental groups (8 mice/group) and three control groups (4 mice/group). Animals were intramuscularly (i.m.) vaccinated with Ad/opt-BoNT/C—H_{C}50 vector (2×10⁷ pfu/mouse) in experimental groups and with Ad/Null (2×10⁷ pfu/mouse) in control groups in week 0. Animal sera were obtained by retro-orbital bleeding in weeks 0, 10, 18, and 26. One experiment and one control group were challenged i.p. with 100×MLD₅₀ BoNT/C in weeks 11, 19, and 27.

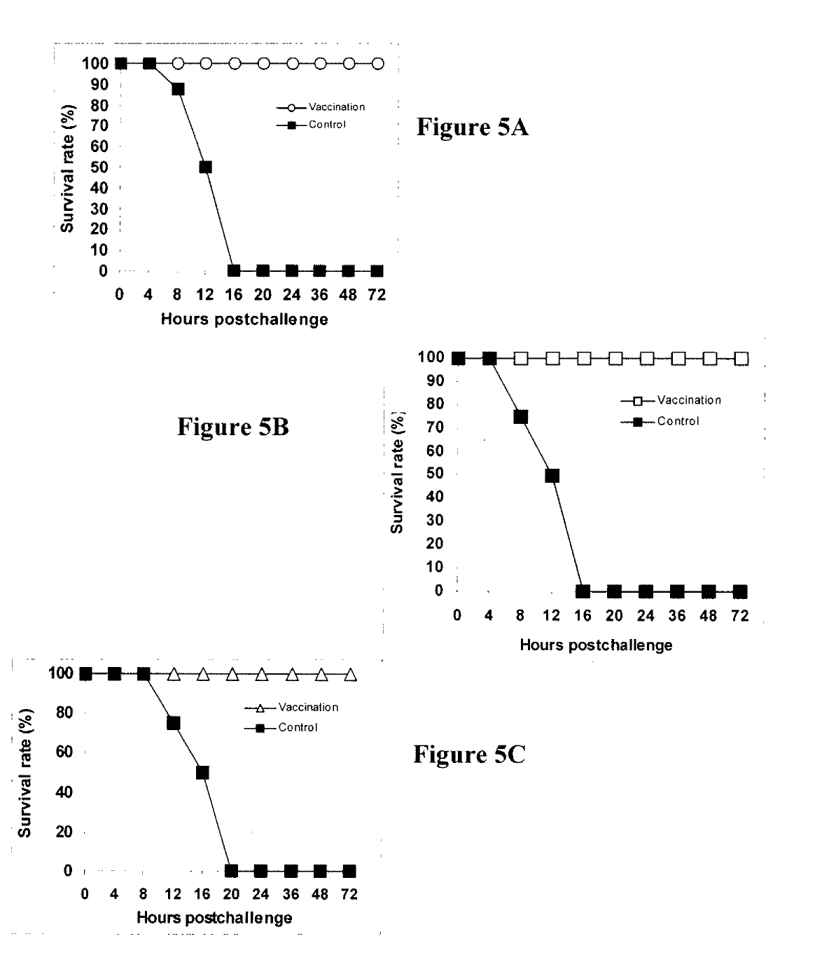

As shown in FIG. 4, BoNT/C—H_{C}50-specific antibody titers in sera were sustained until at least 27 weeks after vaccination (p<0.05). To evaluate protective immunity at different time points, the vaccinated animals were subsequently challenged with BoNT/C neurotoxin in weeks 11, 19, and 27. As indicated in FIGS. 5A-C, a single dose of Ad/opt-BoNT/C—H_{C}50 administered i.m. completely protected animals against botulism at all time points examined. This indicates that long-lasting memory immunity against botulinum neurotoxin was elicited after a single dose of the adenovirus-vectored vaccine.

### Example 5

### Influence of Pre-Existing Anti-Adenovirus Immunity on the Efficacy of Vaccination

An investigation was also made to assess whether host pre-existing immunity to the adenoviral vector could limit the efficacy of the vaccination with this adenovirus-based vaccine.

Twelve mice were allotted into one experiment (8 mice/group) and one control group (4 mice/group). Animals were intranasally (i.n.) inoculated with wide-type human adenovirus serotype 5 (2×10⁷ pfu/mouse) (WT Ad5) (ATCC, VA) in week 0 and bled in week 4. They were subsequently inoculated with Ad/opt-BoNT/C˜H_{C}50 vector (2×10⁷ pfu/mouse) in the experimental group or with Ad/Null (2×10⁷ pfu/mouse) in the control group in week 4, and challenged i.p. with 100×MLD₅₀ BoNT/C in week 11.

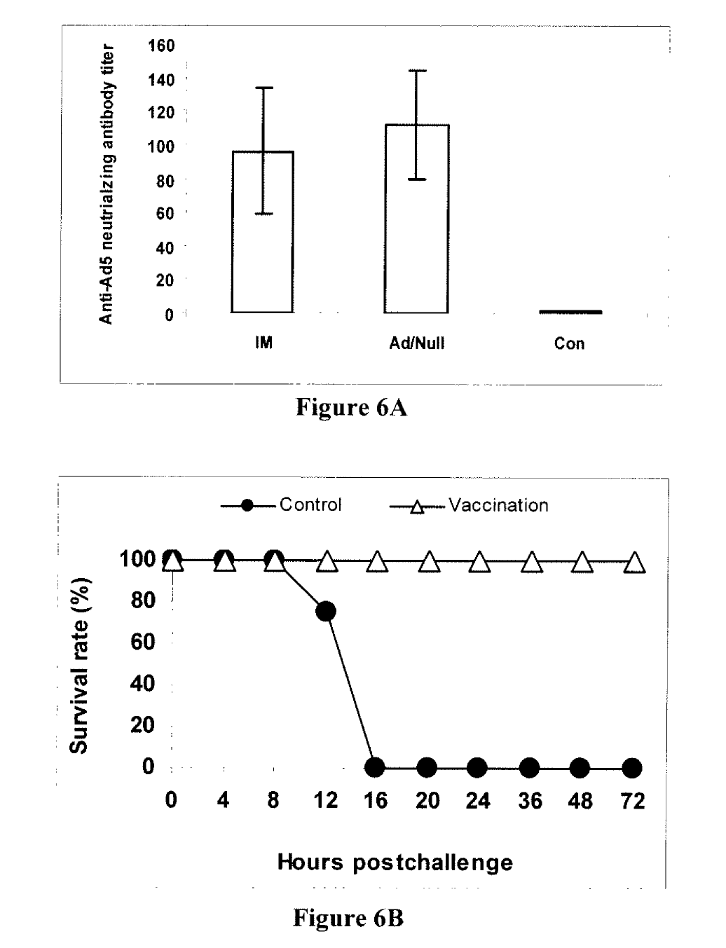

Serum-neutralizing antibody titers against WT Ad5 were assessed by a 96-well neutralization antibody assay described above. The results showed all animals had anti-adenovirus neutralizing antibody responses, with average viral neutralization titers ranging from 96 to 112 (FIG. 6A) 4 weeks after inoculation of WT Ad5. The animals with pre-existing immunity to adenovirus were subsequently inoculated i.m. with the single dose Ad/opt-BoNT/C—H_{C}50 or with control vector Ad/Null in week 4, and challenged in week 11 with BoNT/C neurotoxin. The results in FIG. 6B show that all animals inoculated with Ad/opt-BoNT/C—H_{C}50 survived the toxin challenge and none of the control mice were protected. These data indicate that pre-existing immunity to adenovirus in the host did not affect the protective efficacy of the vaccination with this adenovirus-based botulism vaccine.

### Example 6

### Serum Antibody Responses Against BoNT/C—H_{C}50 Following Intranasal Vaccination

To evaluate an optimal intranasal dose, 40 mice were allotted into 4 experimental groups and one control group, 8 mice per group. The animals were vaccinated by intranasal inhalation with Ad/opt-BoNT/C—H_{C}50 adenoviral vector at doses of 1×10⁴, 1×10⁵, 1×10⁶, and 2×10⁷ pfu/mouse, respectively, in the experimental groups, and Ad/Null at a dose of 2×10⁷ pfu/mouse in the control group. Sera samples were obtained at weeks 0, 2, 4, 6 and stored at −20° C. until further assayed. The experiment mice were i.p. challenged with botulinum neurotoxin one week after the last bleeding as described above.

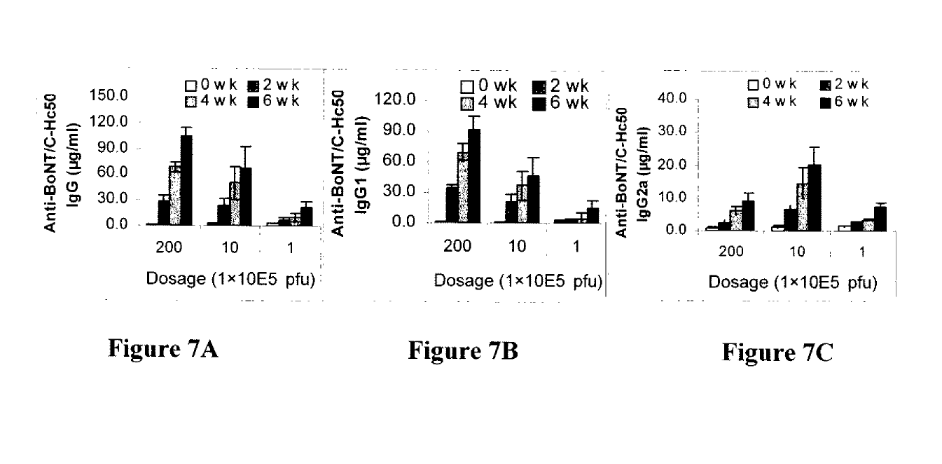

The immune response in sera after a single i.n. vaccination with varying doses adenoviral vector Ad/opt-BoNT/C—H_{C}50 is shown in FIGS. 7A-C. The pre-immune sera (week 0) and sera from mice vaccinated with negative control vector (Ad/Null, adenovirus vector without transgene) were negative. IgG, IgG1 and IgG2a responses to BoNT/C—H_{C}50 were detectable at week 2 for all three doses evaluated. Even at the lowest dose of Ad/opt-BoNT/C—H_{C}50 (1×10⁵ pfu) serum, IgG levels were significantly higher than those of control mice receiving Ad/null (P<0.01). The time-course of the response in serum for IgG, IgG1, and IgG2a to BoNT/C—H_{C}50 in the vaccinated mice is also shown in FIGS. 1A-C. Overall, the dose ranging study showed that the serum IgG concentration in mice receiving 2×10⁷ pfu Ad/opt-BoNT/C—H_{C}50 was the highest (FIG. 7A).

The mice receiving 1×10⁵, 1×10⁶ or 2×10⁷ pfu/mouse of Ad/opt-BoNT/C—H_{C}50 achieved sera anti-BoNT/C—H_{C}50 IgG concentrations of 6.83±2.61, 24.14±7.32, 28.86±6.81 μg/ml respectively at week 2, 9.61±4.87, 50.16±19.11, 68.49±5.58 μg/ml at week 4, 22.59±6.67, 67.20±24.83, 104.98±9.63 μg/ml at week 6. Serum IgG antibodies against BoNT/C—H_{C}50 continued to rise with time from 2 to 6 weeks after vaccination (FIG. 7A).

The IgG1 responses to BoNT/C—H_{C}50 in the mice 6 weeks after vaccination with Ad/opt-BoNT/C—H_{C}50 accounted for about three-fourths of the total IgG. IgG1 concentrations against BoNT/C—H_{C}50 in serum of vaccinated mice were significantly increased by week 2 compared with those in week 0 (FIG. 7B). IgG2a antibody also was produced after vaccination although of lower magnitude than IgG1 (FIG. 7C).

### Example 7

### Persistence of Antibodies after Mucosal Vaccination

In these experiments, 48 mice were allotted into 3 test groups and 3 control groups, 8 mice per group. The animals were i.n. vaccinated at week 0 with Ad/opt-BoNT/C—H_{C}50 vector at doses of 2×10⁷ pfu/mouse in test groups, and Ad/Null at the same dosage in the control groups. One test group and one control group mice were challenged with BoNT/C at weeks 11, 19, and 27, respectively. The serum samples were obtained at week 0 and one week before challenge.

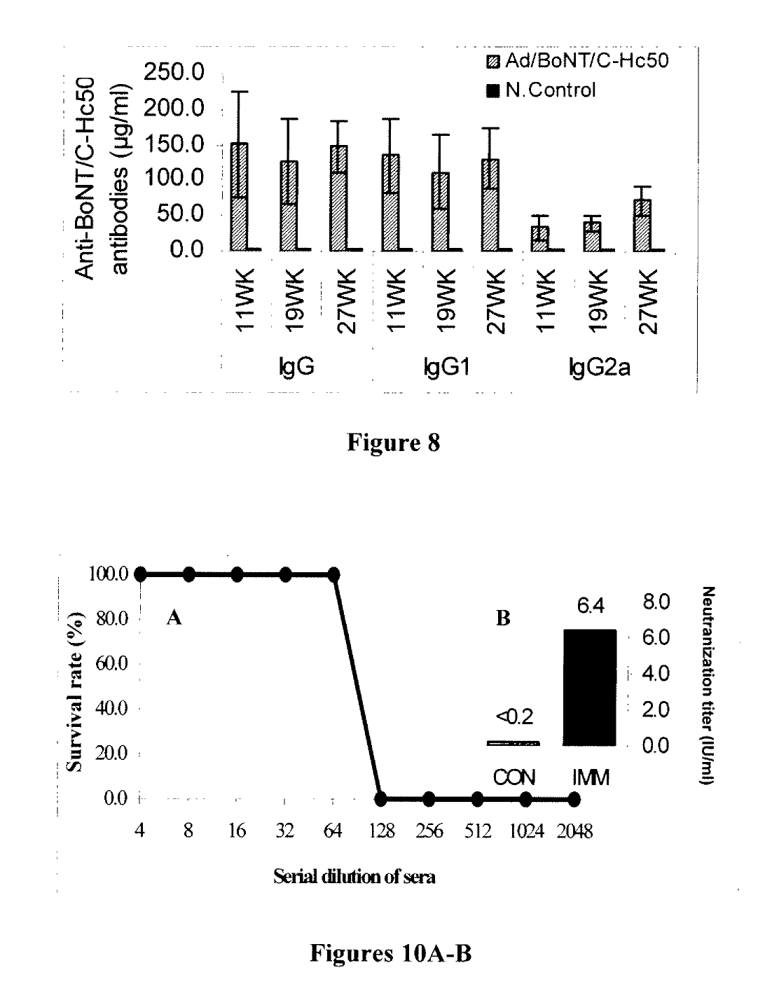

Antibody levels for IgG, IgG1, and IgG2a were measured at 11, 19, and 27 weeks post vaccination (FIG. 8). The results demonstrate that antibody levels persisted at levels similar to post week 6 following vaccination, and they did not significantly decline between week 11 and 27. The same IgG1>IgG2a predominance as observed at post week 6 was observed throughout the experiment. These results for i.n administration are consistent with the results observed in Example 4 for i.p. administration (FIG. 4)

### Example 8

### Antibody Responses against BoNT/C—H_{C}50 in Mucosal Secretions

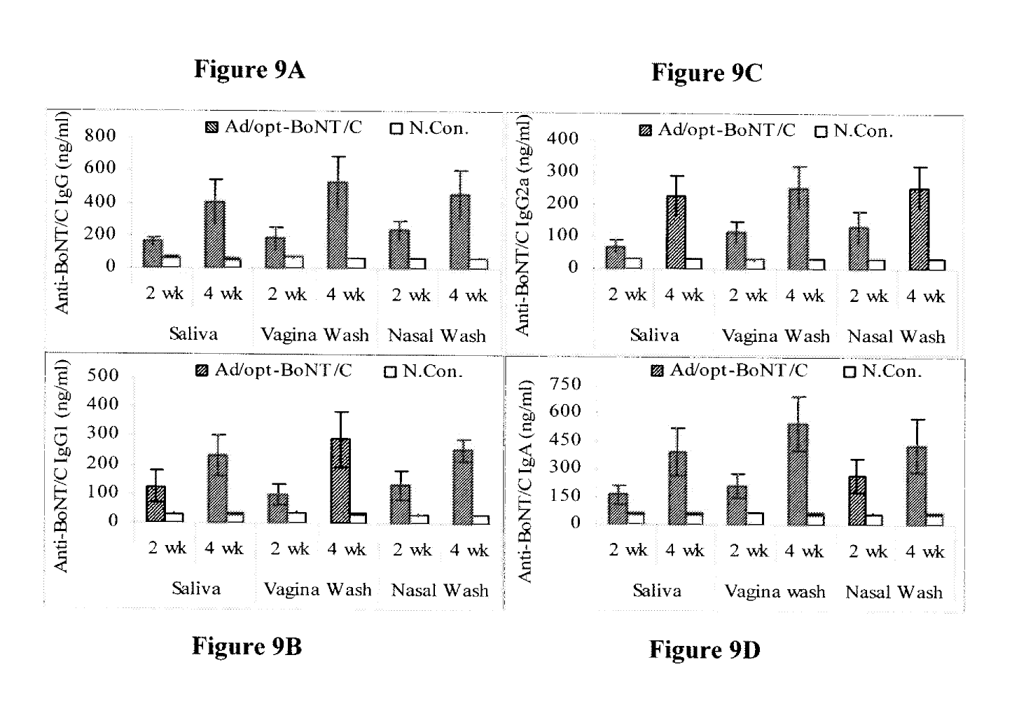

To evaluate the mucosal immune response, specific IgG and IgA antibody concentrations were measured in saliva, nasal wash, and vaginal wash following i.n. vaccination with Ad/opt-BoNT/C—H_{C}50. A single i.n. vaccination with 2×10⁷ pfu/mouse of Ad/opt-BoNT/C—H_{C}50 resulted in significant IgG and IgA antibody responses (FIGS. 9A-D). Two weeks post-vaccination, high local antibody responses were measurable, while no specific antibodies were detectable in the samples from control mice. All the examined antibody levels in the saliva, nasal wash and vaginal wash samples from vaccinated mice at week 4 were significantly higher than those at week 2. The BoNT/C—H_{C}50-specific IgG, IgG1, IgG2a antibody concentrations in mucosal samples were lower than those in sera (P<0.01), and the ratio of IgG2a/IgG1 was reversed compared to that in serum (P<0.01). Mucosal anti-BoNT/C—H_{C}50 IgA in saliva reached 160.4±50 ng/ml at week 2 and 393±132 ng/ml at week 4 (FIG. 9D), while sera anti-BoNT/C—H_{C}50 IgA was not detectable.

### Example 9

### Neutralizing Capacity of Anti-Sera to Botulinum Neurotoxin

Neutralizing antibody titers to BoNT/C were measured by the ability of anti-sera from mice i.n. vaccinated with Ad/opt-BoNT/C—H_{C}50 to neutralize the neurotoxin in vitro. The neutralization capacity of was determined using a mouse bioassay as described in Example 3 above. Due to the limited amount of serum available, the sera from 8 vaccinated mice were pooled.

The results of the assay are shown in FIGS. 10A-B. The neutralization titer from the mice receiving single i.n. doses of 2×10⁷ pfu/mouse of Ad/opt-BoNT/C—H_{C}50 was 6.4 IU/ml 6 weeks after vaccination (FIG. 10b). The antiserum from these mice, diluted by 64-fold or less, completely neutralized 100×MLD₅₀ of BoNT/C resulting in a 100% survival (FIG. 10A). Serum from control mice did not neutralize the neurotoxin.

### Example 10

### Protection against Active BoNT/C in Vaccinated Mice

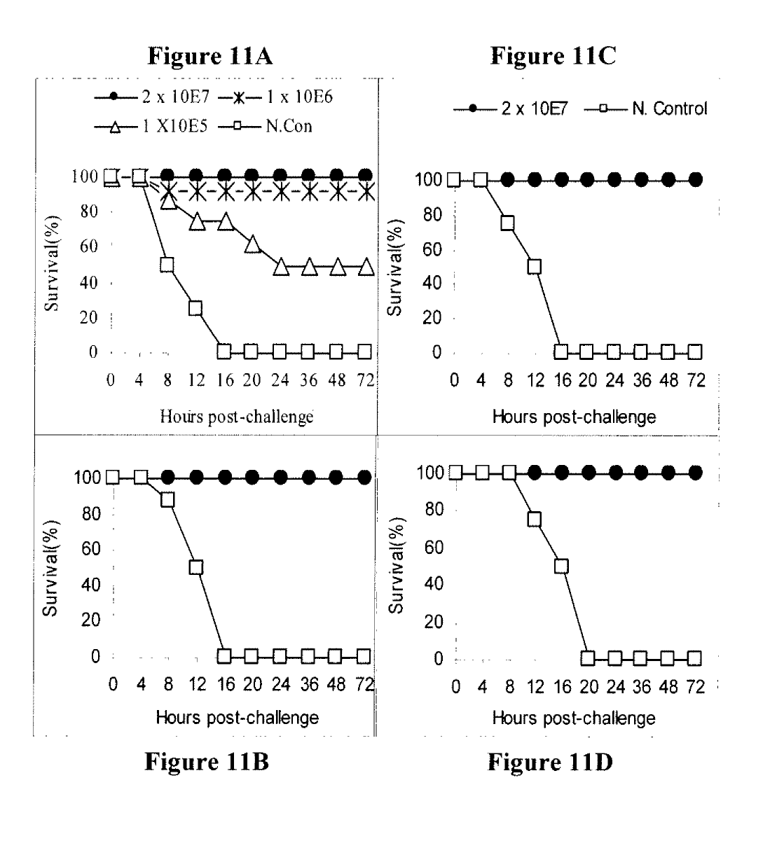

After vaccination intranasally with a single dose of Ad vector, mice were challenged intraperitoneally (i.p.) with 100×MLD₅₀ of active BoNT/C. The results are summarized in FIGS. 11A-D. Seven weeks after a single vaccination none of the mice that received control vector Ad/Null survived the toxin challenge, whereas all mice (8/8, or 100%) that received 2×10⁷ pfu of Ad/opt-BoNT/C—H_{C}50 and 92% mice (11/12) that received 1×10⁶ pfu/mouse of Ad/opt-BoNT/C—H_{C}50 survived 100×MLD₅₀ challenge with no botulism symptoms (FIG. 11A). Fifty-percent (4 of 8 mice) at the lowest vaccine dose studied (1×10⁵ pfu of Ad/opt-BoNT/C—H_{C}50) died (FIG. 11A), and one of the four surviving mice showed botulism symptoms. As shown in FIGS. 11B-D, 11, 19 and 27 weeks after immunization, mice given 2×10⁷ pfu of Ad/opt-BoNT/C—H_{C}50 also were completely protected from 100×MLD₅₀ of BoNT/C toxin.

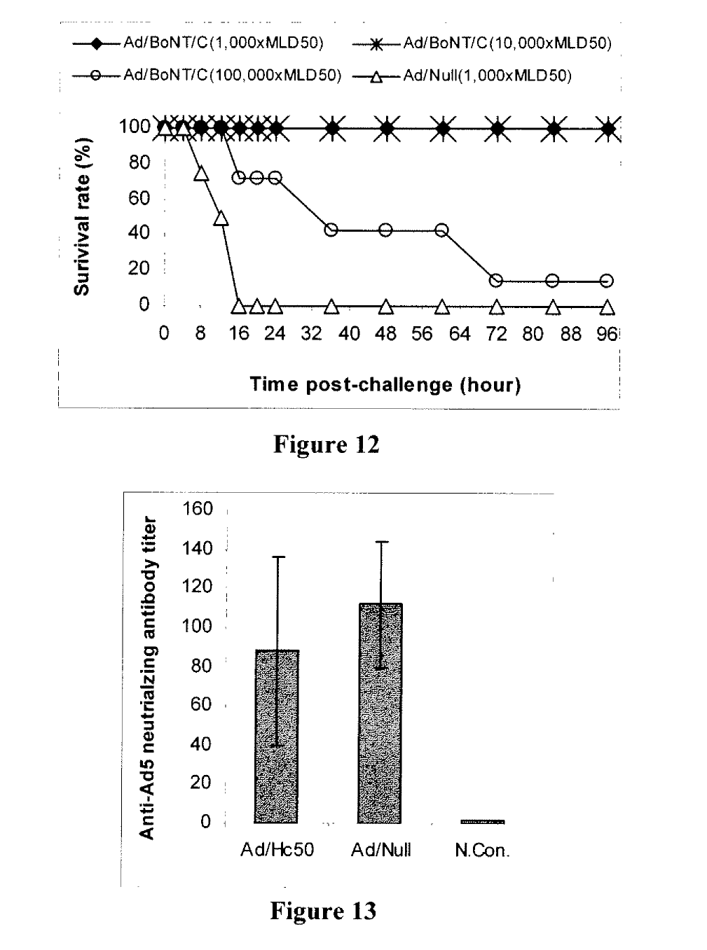

To further assess the vaccine potency for protection, higher doses of active BoNT/C up 10⁵×MLD₅₀ were also used in toxin challenge. FIG. 12 shows that animals i.n. vaccinated with 2×10⁷ pfu of Ad/opt-BoNT/C—H_{C}50 could be completely protected against challenge with 10⁴×MLD₅₀ of BoNT/C—a 100-fold increase in dosage—four weeks after vaccination. However, the protection rate decreased to 14% (1 of 7 mice) when the BoNT/C toxin challenge dose rose to 10⁵×MLD₅₀. This shows the protective immunity, though significant, is also toxin challenge dose dependent.

### Example 11

### Effect of Preexisting Anti-Ad5 Immunity on Vaccination

As in Example 5 above, an assessment was made as to the effect of preexisting anti-human Ad5 neutralization antibody on the efficacy of i.n. vaccination with Ad/opt-BoNT/C—H_{C}50. Twelve mice were allotted into one experimental group (8 mice) and one control group (4 mice). All animals were i.n. inoculated with WT Ad5 at a dose of 2×10⁷ pfu/mouse, and then vaccinated by nasal inhalation at week 4 with Ad/opt-BoNT/C—H_{C}50 vector at a dose of 2×10⁷ pfu/mouse in the experimental group and Ad/Null at the same dose in the control group. The serum samples were obtained at weeks 0, 4, prior to inoculation with WT Ad5 and i.n. vaccination with Ad/opt-BoNT/C—H_{C}50, respectively. The vaccinated animals were subsequently challenged i.p. with 100×MLD₅₀ of BoNT/C at week 11.

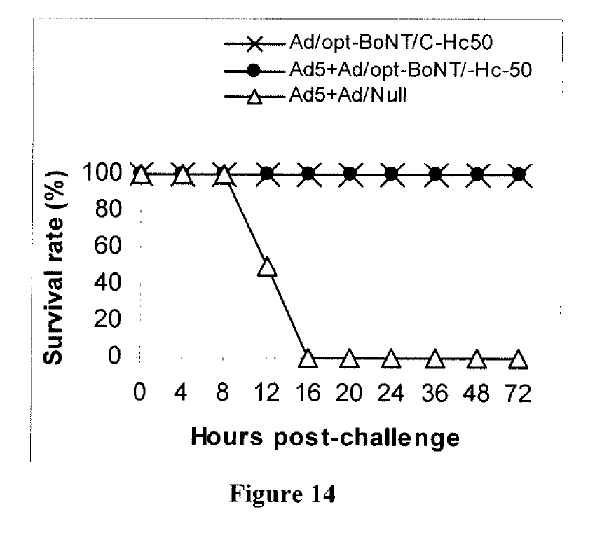

Significant serum anti-Ad5 neutralizing antibody titers were produced (FIG. 13). The results show that all the vaccinated animals were fully protected against 100×MLD₅₀ BoNT/C challenge, whereas none of the control mice survived the toxin challenge (FIG. 14). These data indicate that the Ad/opt-BoNT/C—H_{C}50 vector provides protection against BoNT/C neurotoxin despite pre-existing immunity to adenovirus in the host.

### Discussion of Examples 1-11

The neurotoxins produced by C. botulinum are among the most potent poisons known and there is a need to prepare for their use in a bioterrorism attack (Villar et al., “Botulism: The Many Faces of Botulinum Toxin and Its Potential for Bioterrorism,” Infect Dis Clin North Am 20:313-327 ix, (2006); Atlas R M., “Bioterriorism: From Threat to Reality,” Annu Rev Microbiol 56: 167-185 (2002), each of which is hereby incorporated by reference in its entirety). Botulinum neurotoxins can be lethal by ingestion of minute amounts in food and/or by inhalation. The latter delivery mode is the strongest bioterrorism-related threat (Arnon et al., “Botulinum Toxin as a Biological Weapon,” in Henderson, eds., Bioterrorism: Guidelines for Medical and Public Health Management, Chicago, Ill.: AMA Press, pp. 141-165 (2002); Caya et al., “Clostridium botulinum and the Clinical Laboratorian: A Detailed Review of Botulism, Including Biological Warfare Ramifications of Botulinum Toxin,” Arch Pathol Lab Med 128:653-662 (2004), each of which is hereby incorporated by reference in its entirety). The mucosal immune system is the first line of defense against botulism. However the current injection-type botulism toxoid vaccine only provides protective immunity in the systemic compartment. Clearly, the development of a safe and effective mucosal vaccine should be a high priority against bioterrorism-related botulism (Fujihashi et al., “Mucosal Vaccine Development for Botulinum Intoxication,” Expert Rev Vaccines 6:35-45 (2007); which is hereby incorporated by reference in its entirety).

Protection against botulism neurotoxin is expected to be antibody-mediated and antibody levels have been correlated with protection (Byrne et al., “Fermentation, Purification, and Efficacy of a recombinant Vaccine Candidate against Botulinum Neurotoxin Type F from Pichia pastoris,” Protein Expr Purif 18:327-337 (2000); Holley et al., “Cloning, Expression and Evaluation of a Recombinant Sub-Unit Vaccine Against Clostridium botulinum Type F Toxin,” Vaccine 19:288-297 (2000), each of which is hereby incorporated by reference in its entirety).

The genetic vaccination strategy was previously attempted in botulism vaccine development. Clayton and Middlebrook constructed a plasmid DNA encoding the nontoxic H_{C}50 region of BoNT/A and showed it to partially protect against toxin challenge after up to 4 booster injections (Clayton et al., “Vaccination of Mice with DNA Encoding a Large Fragment of Botulinum Neurotoxin Serotype A,” Vaccine 18(17):1855-62 (2000), which is hereby incorporated by reference in its entirety). Subsequently, in another study using constructed plasmid DNA expressing BoNT/F H_{C} under the control of human ubiquitin gene (UbC) promoter, it was found that two i.m. injections afforded 90% protection against BoNT/F challenge (Jathoul et al., “Efficacy of DNA Vaccines Expressing the Type F Botulinum Toxin Hc Fragment Using Different Promoters,” Vaccine 22(29-30):3942-6 (2004), which is hereby incorporated by reference in its entirety). In addition, Lee and coworkers introduced the H_{C}50 of BoNT/A or BoNT/C into the Venezuelan equine encephalitis (VEE) virus replicon vector, which not only yielded high levels of H_{C} fragments, as judged by immunofluorescence and immunoblotting analysis, but also protected mice against BoNT/A or BoNT/C challenge after two or three injections with 10⁷ infectious units (i.u.) of VEE (Pushko et al., “Replicon-Helper Systems From Attenuated Venezuelan Equine Encephalitis Virus: Expression of Heterologous Genes In Vitro and Immunization Against Heterologous Pathogens in vivo,” Virology 239(2):389-401 (1997); Lee et al., “Candidate Vaccine Against Botulinum Neurotoxin Serotype A Derived From a Venezuelan Equine Encephalitis Virus Vector System,” Infect Immun 69(9):5709-15 (2001); Lee et al., “Multiagent Vaccines Vectored by Venezuelan Equine Encephalitis Virus Replicon Elicits Immune Responses to Marburg Virus and Protection Against Anthrax and Botulinum Neurotoxin in Mice,” Vaccine 24(47-48):6886-92 (2006), each of which is hereby incorporated by reference in its entirety). However, a single dose of genetic vaccine was not fully protective against botulism in these studies.

In contrast to these other studies, the Examples presented above demonstrate that it is possible to develop a highly efficient genetic vaccine against botulism using an adenoviral vector encoding the H_{C}50 fragment of BoNT/C. In the recombinant adenovirus constructed in Example 1, the DNA sequence encoding the H_{C}50 fragment antigen was codon-optimized with human codon preference. This resulted in high-level expression of H_{C}50 in mice.

Because of the presence of a signal peptide from human tissue plasminogen activator (PLAT), it is believed that the H_{C}50 fragment antigen was secreted efficiently from adenoviral vector transformed host cells (Ertl et al., “Technical Issues in Construction of Nucleic Acid Vaccines,” Methods 31(3):199-206 (2003); Hermanson et al., “A Cationic Lipid-Formulated Plasmid DNA Vaccine Confers Sustained Antibody-Mediated Protection Against Aerosolized Anthrax Spores,” Proc Natl Acad Sci USA 101(37):13601-6 (2004), each of which is hereby incorporated by reference in its entirety). Consequently, the secretory H_{C}50 was likely presented to antigen-presenting cells (APCs) more efficiently after vaccination.

Examples 1-5 demonstrate that a single i.m. dose of the Ad/opt-BoNT/C—H_{C}50 vector was capable of eliciting significant Th2 and Th1 immune responses against H_{C}50 fragment of BoNT/C (FIGS. 1A-C) and the serum antigen-specific antibodies were capable of neutralizing active BoNT/C (FIGS. 2A-B). The protective antibodies against H_{C}50 of BoNT/C were sustained for long-term, at least up to 27 weeks (FIG. 4) and likely for much longer. Host immune responses and protective immunity appeared to be vaccine dose-dependent. Most importantly, a single dose of 2×10⁷ pfu of Ad/opt-BoNT/C—H_{C}50 was sufficient to provide long-term protective immunity (FIGS. 5A-C). This study is the first to demonstrate that a single genetic vaccination is able to provide long-lasting protection against botulism.

Examples 6-11 extend these initial results, demonstrating that a single i.n. dose of the Ad/opt-BoNT/C—H_{C}50 vector was also capable of eliciting a significant immune response against the H_{C}50 fragment of BoNT/C (FIGS. 7A-C) and the serum antigen-specific antibodies were capable of neutralizing active BoNT/C (FIGS. 10A-B). The protective antibodies against H_{C}50 of BoNT/C were sustained for long-term, at least up to 27 weeks (FIG. 8) and likely for much longer. Host immune responses and protective immunity appeared to be vaccine dose-dependent. Most importantly, a single i.n. dose of 2×10⁷ pfu of Ad/opt-BoNT/C—H_{C}50 was sufficient to provide long-term protective immunity even against high doses of neurotoxin (FIGS. 11A-D, 12).

Because of the simple intranasal route of vaccination, this vaccine can be self-administered to protect the population in the event of terrorist attack with C. botulism or neurotoxins. In addition to the ease of administration and rapid onset of protection demonstrated in the Examples, the vaccine can be produced inexpensively, in high quantity, and in a short time frame. The H_{C}50 fragment of botulinum neurotoxin type C was selected, because the H_{C}50 subunits of BoNTs are non-toxic and antigenic and capable of eliciting immunity responses against botulism (Byrne et al., “Development of Vaccines for Prevention of Botulism,” Biochimie 82:955-966 (2000); Webb et al., “Protection with Recombinant Clostridium botulinum C1 and D Binding Domain Subunit (Hc) Vaccines Against C and D Neurotoxins,” Vaccine 25:4273-4282 (2007); Byrne et al., “Purification, Potency, and Efficacy of the Botulinum Neurotoxin Type A Binding Domain from Pichia pastoris as a Recombinant Vaccine Candidate,” Infect Immun 66:4817-4822 (1998); Atassi et al., “Structure, Activity, and Immune (T and B cell) Recognition of Botulinum Neurotoxins,” Crit Rev Immunol 19:219-260 (1999), each of which is hereby incorporated by reference in its entirety).

Adenoviruses invade their host naturally via the mucosa surface, notably in the respiratory or gastrointestinal tract (Lemiale et al., “Enhanced Mucosal Immunoglobulin A Response of intranasal Adenoviral Vector Human Immunodeficiency Virus Vaccine and Localization in the Central Nervous System,” J Virol 77:10078-10087 (2003), which is hereby incorporated by reference in its entirety). Adenoviral vector vaccines can be effectively delivered by intranasal mucosal route and can induce strong adaptive immune responses in mammalian hosts (Bangari et al., “Development of Nonhuman Adenoviruses as Vaccine Vectors,” Vaccine; 24:849-862 (2006); Tatsis et al., “Adenoviruses as Vaccine Vectors,” Mol Ther 10:616-629 (2004), each of which is hereby incorporated by reference in its entirety.)

Other studies have established that preexisting anti-adenovirus antibody may drastically reduce the take of adenovirus vectored vaccines (Bangari et al., “Development of Nonhuman Adenoviruses as Vaccine Vectors,” Vaccine; 24:849-862 (2006); Casimiro et al., “Comparative Immunogenicity in Rhesus Monkeys of DNA Plasmid, Recombinant Vaccinia Virus, and Replication-Defective Adenovirus Vectors Expressing a Human Immunodeficiency Virus Type 1 Gag Gene,” J Virol 77:6305-6313 (2003); Yang et al., “Cellular and Humoral Immune Responses to Viral Antigens Create Barriers to Lung-Directed Gene Therapy with Recombinant Adenoviruses,” J Virol 69:2004-2015 (1995); Barouch et al., “Plasmid Chemokines and Colony-Stimulating Factors Enhance the Immunogenicity of DNA Priming-Viral Vector Boosting Human Immunodeficiency Virus Type 1 Vaccines,” J Virol 77:8729-8735 (2003), each of which is hereby incorporated by reference in its entirety), but immunity to the vector has been overcome in some situations (Babiuk et al., “Adenoviruses as Vectors for Delivering Vaccines to Mucosal Surfaces,” J Biotechnol 83:105-113 (2000); Papp et al., “The Effect of Pre-Existing Adenovirus-Specific Immunity on Immune Responses Induced by Recombinant Adenovirus Expressing Glycoprotein D of Bovine Herpesvirus Type 1,” Vaccine 17:933-943 (1999); Fischer et al., “Vaccination of Puppies Born to immune Dams with a Canine Adenovirus-Based Vaccine Protects Against a Canine Distemper Virus Challenge,” Vaccine 20:3485-3497 (2002), each of which is hereby incorporated by reference in its entirety. The data from Examples 5 and 11 demonstrate that even with pre-existing anti-adenovirus neutralizing antibody in the host, the protective efficacy of the vaccination was sustained (FIGS. 6A-B; 13-14). This will, of course, need to be further evaluated in human trials, because the ability for replication of human adenovirus is limited in murine cells (Duncan et al., “Infection of Mouse Liver by Human Adenovirus Type 5,” J Gen Virol 40(1):45-61 (1978); which is hereby incorporated by reference in their entirety) and human immune response to adenovirus may differ from that of mouse.

To avoid the possibility of contamination of replication-competent adenovirus, in future human clinical trials the vector will be prepared using new suitable packaging cell lines such as the Per.C6 and UR cell lines that were developed recently (Fallaux et al., “New Helper Cells and Matched Early Region 1-Deleted Adenovirus Vectors Prevent Generation of Replication-Competent Adenoviruses,” Hum Gene Ther 9(13):1909-17 (1998); Schiedner et al., “Efficient Transformation of Primary Human Amniocytes by E1 Functions of Ad5: Generation of New Cell Lines for Adenoviral Vector Production,” Hum Gene Ther 11(15):2105-16 (2000); Xu et al., “A New Complementing Cell Line for Replication-Incompetent E1-Deleted Adenovirus Propagation,” Cytotechnology 51:133-40 (2006), each of which is hereby incorporated by reference in its entirety).

In summary, the preceding Examples demonstrate for the first time that an adenovirus-based vector encoding a humanized H_{C}50-kDa fragment of BoNT/C is capable of eliciting robust host immunity against botulism caused by BoNT/C after a single dose regardless of the mode of administration. Both intramuscular and intranasal administration elicited high serum antibody response to BoNTC/H_{C}50. The anti-BoNT/C protective immunity generated by these vaccinations was sustained for a prolonged time period. This strategy can also be applied for the development of a multivalent vaccine against all serotypes of botulinum neurotoxins.

### Example 12

### Construction of Nucleic Acid Molecule Encoding Codon-Optimized H_{C}50 of BoNT/A Chimeric Protein, and Insertion in Adeno-viral Vector

An adenoviral vector encoding human codon-optimized H_{C}50 of BoNT/A was synthesized using the same strategy described above for construction of Ad/opt-BoNT/C—H_{C}50. The codon-optimized H_{C}50 of BoNT/A nucleotide sequence (SEQ ID NO: 5) is shown in Table 2 above.

The adenoviral vector prepared above will be screened via intramuscular injection and intranasal instillation to mice for the generation of an immune response against BoNT/A. The antibody titer generated and the ability of the antisera to neutralize BoNT/A toxicity will be assessed as demonstrated in the preceding Examples. The generation of long term protection (longer than 6 months) will also be assessed.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An isolated DNA molecule comprising a first segment encoding a fragment of a heavy chain region of a Clostridium botulinum neurotoxin, wherein the first segment is codon-enhanced to improve expression of the isolated DNA molecule in a mammalian host.

2. The isolated DNA molecule according to claim 1, wherein the mammalian host is a human or a non-human primate.

3. The isolated DNA molecule according to claim 1, wherein the Clostridium botulinum neurotoxin is neurotoxin A, neurotoxin B, neurotoxin C, neurotoxin D, neurotoxin E, or neurotoxin F, or neurotoxin G.

4. The isolated DNA molecule according to claim 1, wherein the Clostridium botulinum neurotoxin is neurotoxin C or neurotoxin A.

5. The isolated DNA molecule according to claim 4, wherein the fragment of the heavy chain region of Clostridium botulinum neurotoxin C or neurotoxin A comprises a C-terminal fragment that is about 50 kDa.

6. The isolated DNA molecule according to claim 4, wherein the nucleotide sequence of the first segment is SEQ ID NO: 2 or SEQ ID NO: 5.

7.-9. (canceled)

10. The isolated DNA molecule according to claim 1, wherein the DNA molecule includes a second segment located 5′ to the first segment, the second segment encoding a secretion signal peptide comprising a secretion signal from human tissue plasminogen activator, human serum albumin, human IL-3, or human growth hormone.

11. (canceled)

12. An expression vector comprising the isolated DNA molecule according to claim 1 operably coupled to a promoter sequence located 5′ to the isolated DNA molecule and a transcription termination sequence located 3′ to the isolated DNA molecule.

13. (canceled)

14. The expression vector according to claim 12, wherein the promoter sequence is a constitutive promoter.

15.-16. (canceled)

17. The expression vector according to claim 12, wherein the expression vector is a replication-defective adenoviral vector.

18. The expression vector according to claim 12, wherein the expression vector comprises two or more isolated DNA molecules encoding fragments of different Clostridium botulinum neurotoxins.

19. A host cell comprising the expression vector according to claim 12.

20. (canceled)

21. The host cell according to claim 19, wherein the host cell is in vivo.

22. The host cell according to claim 19, wherein the host cell is a mammalian cell.

23. (canceled)

24. A chimeric protein comprising a secretion signal peptide linked N-terminal of a fragment of a heavy chain region of a Clostridium botulinum neurotoxin.

25. The chimeric protein according to claim 24, wherein the secretion signal peptide comprises a secretion signal from human tissue plasminogen activator, human serum albumin, human IL-3, or human growth hormone.

26. The chimeric protein according to claim 24, wherein the Clostridium botulinum neurotoxin is neurotoxin A, neurotoxin B, neurotoxin C, neurotoxin D, neurotoxin E, or neurotoxin F, or neurotoxin G.

27. (canceled)

28. The chimeric protein according to claim 26, wherein the fragment of the heavy chain region of a Clostridium botulinum neurotoxin comprises a C-terminal fragment of neurotoxin C or neurotoxin A that is about 50 kDa.

29. The chimeric protein according to claim 28, wherein the C-terminal fragment of the heavy chain region of Clostridium botulinum neurotoxin C or neurotoxin A comprises the amino acid sequence of SEQ ID NO: 3 or SEQ ID NO: 6.

30.-32. (canceled)

33. The chimeric protein according to claim 24, wherein the secretion signal peptide comprises the amino acid sequence of SEQ ID NO: 7 and the fragment of the heavy chain region of Clostridium botulinum neurotoxin comprises the amino acid sequence of SEQ ID NO: 3 or SEQ ID NO: 6.

34. The chimeric protein according to claim 33, wherein the chimeric protein further comprises a linker sequence between the secretion signal peptide and the fragment of the heavy chain region of Clostridium botulinum neurotoxin.

35. (canceled)

36. A vaccine comprising a pharmaceutically acceptable carrier and either (i) a DNA molecule comprising a first segment encoding a fragment of a heavy chain region of a Clostridium botulinum neurotoxin, wherein the first segment is codon-enhanced to improve expression of the isolated DNA molecule in a mammalian host, or (ii) a chimeric protein comprising a secretion signal peptide linked N-terminal of a fragment of a heavy chain region of a Clostridium botulinum neurotoxin, or a combination thereof.

37. (canceled)

38. A method of imparting resistance against a Clostridium botulinum neurotoxin to a mammal comprising:
administering a vaccine according to claim 36 to a mammal under conditions effective to induce a protective immune response against the Clostridium botulinum neurotoxin.

39. The method according to claim 38, wherein said administering is carried out orally, parenterally, subcutaneously, intravenously, intramuscularly, intraperitoneally, by intranasal instillation, by implantation, by intracavitary or intravesical instillation, intraarterially, intralesionally, transdermally, by application to mucous membranes.

40.-41. (canceled)

42. The method according to claim 38, wherein the vaccine comprises the DNA molecule.

43. The method according to claim 38, wherein the vaccine comprises the chimeric protein, the chimeric protein comprising a secretion signal peptide comprising the amino acid sequence of SEQ ID NO: 7 and a fragment of the heavy chain region of Clostridium botulinum neurotoxin comprising the amino acid sequence of SEQ ID NO: 3 or SEQ ID NO: 6.

44.-47. (canceled)

48. An isolated antibody raised against a chimeric protein according to claim 24, or binding fragment thereof.

49.-52. (canceled)

53. A pharmaceutical composition comprising a pharmaceutically acceptable carrier and an antibody, or binding fragment thereof, according to claim 48.

54. (canceled)

55. A method of treating a Clostridium botulinum infection comprising administering to a patient an antibody or antibody fragment thereof according to claim 48, under conditions effective to neutralize a botulism neurotoxin, and thereby treat the Clostridium botulinum infection.
